(19) United States
(12) Patent Application Publication
(10) Pub. No.: US 2017/0196989 A1
(43) Pub. Date: Jul. 13, 2017

(54) RADIATION-STERILIZED BIODEGRADABLE DRUG DELIVERY COMPOSITIONS

(71) Applicant: Durect Corporation, Cupertino, CA (US)

(72) Inventors: Michael Sekar, Palo Alto, CA (US); Su Il Yum, Los Altos, CA (US); Felix Theeuwes, Los Altos Hills, CA (US); William W. van Osdol, Mountain View, CA (US)

(73) Assignee: Durect Corporation, Cupertino, CA (US)

(21) Appl. No.: 15/384,087

(22) Filed: Dec. 19, 2016

Related U.S. Application Data
(63) Continuation of application No. 14/360,297, filed on May 22, 2014, now abandoned.
(60) Provisional application No. 61/563,469, filed on Nov. 23, 2011.

(51) Int. Cl. A61K 47/48; A61K 9/00; A61K 9/16; A61K 9/19; A61K 38/26; A61L 2/00; A61K 38/27; A61K 31/7052; A61K 47/34

(52) U.S. Cl. CPC A61K 47/48246; A61K 38/27; A61K 47/48015; A61K 9/0019; A61K 9/1623; A61K 9/1617; A61K 9/1647; A61K 9/19; A61K 9/16; A61K 31/7052; A61K 38/26; A61L 2/0035; A61L 2/007; A61L 2/0041; A61L 2202/21

(57) ABSTRACT

The present disclosure is directed to a method of making a composition by combining a vehicle, e.g., a single phase vehicle, and an insoluble component comprising a beneficial agent, and sterilizing the composition using ionizing radiation prior to use, wherein the beneficial agent is stable following exposure to a sterilizing dose of ionizing radiation. Related compositions and methods are provided.

### CROSS-REFERENCE TO RELATED APPLICATIONS

This application claims the benefit of U.S. Provisional Patent Application No. 61/563,469 filed Nov. 23, 2011, which application is incorporated herein by reference in its entirety.

The present application expressly incorporates by reference herein the entire disclosure of U.S. application Ser. No. 13/304,174, entitled, “Biodegradable Drug Delivery Composition,” Attorney Docket No. DURE-076, filed Nov. 23, 2011, which was incorporated by reference by the above-noted U.S. Provisional Patent Application No. 61/563,469.

### INTRODUCTION

A variety of compositions designed for the delivery of beneficial agent are available which utilize various combinations of polymers, solvents and other components. However, many of these compositions are not suitable for sterilization using ionizing radiation, e.g., gamma radiation, e-beam radiation or x-ray radiation, which is an important consideration for pharmaceutical production. Where ionizing radiation is not suitable, processing compositions under aseptic conditions is expensive and not always successful. In addition to the above sterility considerations, compositions having good syringeability and injectability that are suitable for use with narrow gauge needles or needleless injectors are an important consideration, e.g., for reducing pain at the time of injection. The present disclosure addresses these issues and provides related advantages.

### SUMMARY

The present disclosure is directed to a method of making a composition by combining a vehicle, e.g., a single phase vehicle, and an insoluble component comprising a beneficial agent, and sterilizing the composition using ionizing radiation prior to use, wherein the beneficial agent is stable following exposure to a sterilizing dose of ionizing radiation. Related compositions and methods are provided.

Certain non-limiting aspects of the disclosure are provided below:
1. A composition, comprising:
   a single-phase vehicle, comprising:
      a biodegradable polymer in an amount ranging from about 5% to about 40% by weight of the vehicle, and
      a hydrophobic solvent in an amount ranging from about 95% to about 60% by weight of the vehicle; and
   an insoluble beneficial agent complex comprising a beneficial agent in the vehicle, wherein the composition has been irradiated with ionizing radiation, and wherein the beneficial agent is present at a purity of about 90% or greater.
2. The composition of 1, wherein the ionizing radiation is selected from gamma radiation, e-beam radiation and x-ray radiation.
3. The composition of any one of the above, wherein the beneficial agent maintains a purity of about 90% or greater when stored at 25° C. for a period of one month.
4. The composition of any one of the above, wherein the ionizing radiation comprises a dose of about 10 kGy to about 25 kGy.
5. The composition of any one of the above, wherein the insoluble beneficial agent complex comprises a peptide or a protein as the beneficial agent.
6. The composition of any one of the above, wherein the ionizing radiation is gamma radiation.
7. The composition of any one of the above, wherein the composition has a zero shear viscosity less than 1,200 centipoise at 25° C. and is not an emulsion, a gel or gel forming.
8. The composition of any one of the above, wherein the composition has a zero shear viscosity less than 1,000 centipoise at 25° C.
9. The composition of any one of the above, wherein the composition has a zero shear viscosity less than 500 centipoise at 25° C.
10. The composition of any one of the above, wherein the composition has a zero shear viscosity less than 100 centipoise at 25° C.
11. The composition of any one of the above, wherein the composition has a zero shear viscosity of less than 1200 centipoise and greater than 10 centipoise at 25° C.
12. The composition of any one of the above, wherein the insoluble beneficial agent complex comprises protamine.
13. The composition of any one of the above, further comprising an antioxidant.
14. The composition of 13, wherein the antioxidant is present in an amount ranging from about 1 wt % to about 45 wt %, relative to the amount of beneficial agent.
15. The composition of any one of the above, further comprising methionine.
16. The composition of 15, wherein the methionine is present in an amount ranging from about 0.1 wt % to about 45 wt %, relative to the amount of beneficial agent.
17. The composition of 16, wherein the methionine is present in an amount ranging from about 1 wt % to about 45 wt % of the beneficial agent.
18. The composition of any one of the above, wherein the insoluble beneficial agent complex comprises a beneficial agent having a molecular weight greater than 5 kD.
19. The composition of any one of the above, wherein the insoluble beneficial agent complex comprises a beneficial agent having a molecular weight greater than 10 kD.
20. The composition of any one of the above, wherein the insoluble beneficial agent complex comprises a beneficial agent having a molecular weight greater than 10 kD and less than 1000 kD.
21. The composition of any one of the above, wherein the insoluble beneficial agent complex comprises a divalent metal salt of the beneficial agent.
22. The composition of 21, wherein the divalent metal is selected from Zn²⁺, Mg²⁺ and Ca²⁺.
23. The composition of 22, wherein the divalent metal is Zn²⁺.
24. The composition of any one of the above, wherein the insoluble beneficial agent complex comprises beneficial agent and protamine in the form of particles, and wherein the particles further comprise bulking agent and surfactant.
25. The composition of any one of the above, wherein the insoluble beneficial agent complex comprises a beneficial agent, Zn²⁺ and protamine at a molar ratio of approximately 1:0.5 to 2.0:0.3 to 0.5.
26. The composition of any one of 1 to 24, wherein the insoluble beneficial agent complex comprises a beneficial agent and protamine, and wherein the molar ratio of the beneficial agent and protamine is approximately 1:0.1 to 0.5.
27. The composition of any one of the above, wherein the insoluble beneficial agent complex is dispersed in the vehicle in the form of particles having an average size ranging from about 1 μm to about 400 μm.
28. The composition of any one of the above, wherein the insoluble beneficial agent complex is dispersed in the vehicle in the form of particles having an average size ranging from about 1 μm to about 100 μm.
29. The composition of any one of 27 and 28, wherein the particles comprise freeze-dried particles.
30. The composition of any one of 1 to 26, wherein the insoluble beneficial agent complex is dispersed in the vehicle in the form of particles having an average size ranging from about 1 μm to about 10 μm.
31. The composition of 30, wherein the particles comprise spray-dried particles.
32. The composition of any one of the above, wherein when 0.8 mL of the composition is placed in a 1 mL syringe at 25° C. fitted with a 0.5 inch long needle with a gauge of 21 and 10 lbs of force are applied, at least 0.5 mL of the composition is ejected from the syringe in less than 10 seconds, and wherein the composition is not an emulsion.
33. A method of making a composition, comprising:
   combining a biodegradable polymer and a hydrophobic solvent to form a single-phase vehicle of the composition,
      wherein the biodegradable polymer is included in an amount of from about 5% to about 40% by weight of the vehicle, and
      the hydrophobic solvent is included in an amount of from about 95% to about 60% by weight of the vehicle;
   dispersing an insoluble beneficial agent complex comprising a beneficial agent in the vehicle to form the composition; and
   irradiating the composition with ionizing radiation, wherein the beneficial agent maintains a purity of about 90% or greater when stored at 25° C. for a period of 24 hours after irradiation.
34. The method of 33, wherein the ionizing radiation is selected from gamma radiation, e-beam radiation and x-ray radiation.
35. The method of any one of 33 to 34, wherein the beneficial agent maintains a purity of about 90% or greater when stored at 25° C. for a period of one month.
36. The method of any one of 33 to 35, wherein the irradiating comprises exposing the composition to ionizing radiation at a dose of about 10 kGy to about 25 kGy.
37. The method of any one of 33 to 36, wherein the insoluble beneficial agent complex comprises a peptide or a protein as the beneficial agent.
38. The method of any one of 33 to 37, wherein the beneficial agent maintains a purity of about 95% or greater when stored at 25° C. for a period of 24 hours following exposure to ionizing radiation at a dose of about 15 kGy.
39. The method of any one of 33 to 38, wherein the ionizing radiation is gamma radiation.
40. The method of any one of 33 to 39, wherein the beneficial agent maintains a purity of about 95% or greater when stored at 25° C. for a period of 24 hours following exposure to ionizing radiation at a dose of about 25 kGy.
41. The method of 40, wherein the ionizing radiation is gamma radiation.
42. The method of 40 or 41, wherein the beneficial agent maintains a purity of about 95% or greater when stored at 25° C. for a period of one month.
43. The method of any one of 33 to 42, wherein the irradiating is conducted at from about 2° C. to 8° C.
44. The method of any one of 33 to 42, wherein the irradiating is conducted at from 0° C. to 30° C.
45. The method of any one of 33 to 44, wherein the composition has a zero shear viscosity less than 1,200 centipoise at 25° C. and is not an emulsion, a gel or gel forming.
46. The method of any one of 33 to 45, wherein the composition has a zero shear viscosity less than 1,000 centipoise at 25° C.
47. The method of any one of 33 to 46, wherein the composition has a zero shear viscosity less than 500 centipoise at 25° C.
48. The method of any one of 33 to 47, wherein the composition has a zero shear viscosity less than 100 centipoise at 25° C.
49. The method of any one of 33 to 48, wherein the composition has a zero shear viscosity of less than 1200 centipoise and greater than 10 centipoise at 25° C.
50. The method of any one of 33 to 49, wherein the insoluble beneficial agent complex comprises protamine.
51. The method of any one of 33 to 50, further comprising adding antioxidant to the composition prior to irradiating the composition.
52. The method of 51, wherein the antioxidant is added in an amount ranging from about 1 wt % to about 45 wt %, relative to the amount of beneficial agent.
53. The method of any one of 33 to 50, further comprising adding methionine to the composition prior to irradiating the composition.
54. The method of 53, wherein the methionine is added in an amount ranging from about 0.1 wt % to about 45 wt %, relative to the amount of beneficial agent.
55. The method of any one of 53 to 54, wherein the methionine is added in an amount from about 1 wt % to about 45 wt % of the beneficial agent.
56. The method of any one of 33 to 55, wherein the insoluble beneficial agent complex comprises a beneficial agent having a molecular weight greater than 5 kD.
57. The method of any one of 33 to 56, wherein the insoluble beneficial agent complex comprises a beneficial agent having a molecular weight greater than 10 kD.
58. The method of any one of 33 to 57, wherein the insoluble beneficial agent complex comprises a beneficial agent having a molecular weight greater than 10 kD and less than 1000 kD.
59. The method of any one of 33 to 58, wherein the insoluble beneficial agent complex comprises a divalent metal salt of the beneficial agent.
60. The method of 59, wherein the divalent metal is selected from Zn²⁺, Mg²⁺ and Ca²⁺.
61. The method of 59, wherein the divalent metal is Zn²⁺.
62. The method of any one of 33 to 61, further comprising forming the insoluble beneficial agent complex by combining the beneficial agent with protamine, wherein the method further comprises combining the insoluble beneficial agent complex with bulking agent and surfactant to form particles prior to irradiating the composition.
63. The method of any one of 33 to 62, comprising forming the insoluble beneficial agent complex by combining a beneficial agent, Zn²⁺ and protamine at a molar ratio of approximately 1:0.5 to 2.0:0.3 to 0.5.
64. The method of any one of 33 to 62, further comprising forming the insoluble beneficial agent complex by combining a beneficial agent and protamine, wherein the molar ratio of the beneficial agent and protamine is approximately 1:0.1 to 0.5.
65. The method of any one of 33 to 64, comprising dispersing the insoluble beneficial agent complex in the vehicle in the form of particles having an average size ranging from about 1 μm to about 400 μm.
66. The method of any one of 33 to 65, comprising dispersing the insoluble beneficial agent complex in the vehicle in the form of particles having an average size ranging from about 1 μm to about 100 μm.
67. The method of any one of 65 to 66, comprising forming the particles by freeze-drying.
68. The method of any one of 33 to 65, wherein the insoluble beneficial agent complex is dispersed in the vehicle in the form of particles having an average size ranging from about 1 μm to about 10 μm.
69. The method of 68 comprising forming the particles by spray-drying.
70. The method of any one of 33 to 69, wherein when 0.8 mL of the composition is placed in a 1 mL syringe at 25° C. fitted with a 0.5 inch long needle with a gauge of 21 and 10 lbs of force are applied, at least 0.5 mL of the composition is ejected from the syringe in less than 10 seconds, and wherein the composition is not an emulsion.
71. The method of 70, wherein the composition is ejected from the syringe in less than 5 seconds.
72. The method of any one of 33 to 71, comprising milling and sieving the insoluble beneficial agent complex prior to the dispersing.
73. The method of any one of 33 to 72, wherein the irradiating occurs prior to the dispersing.
74. The method of any one of 33 to 72, wherein the irradiating occurs after the dispersing.
75. A method of administering a beneficial agent to a subject, comprising:
   administering to the subject via injection a sterile, irradiated composition comprising
   a vehicle comprising
      a biodegradable polymer present in an amount of from about 5% to about 40% by weight of the vehicle, and
      a hydrophobic solvent present in an amount of from about 95% to about 60% by weight of the vehicle; and
   an insoluble beneficial agent complex dispersed in the vehicle,
   wherein the composition has a zero shear viscosity less than 1,200 centipoise at 25° C. and is not an emulsion, and
   wherein the beneficial agent has a purity of at least 90% or greater.
76. The method of 75, wherein the composition has a zero shear viscosity less than 1,000 centipoise at 25° C.
77. The method of 76, wherein the composition has a zero shear viscosity less than 500 centipoise at 25° C.
78. The method of 77, wherein the composition has a zero shear viscosity less 100 centipoise at 25° C.
79. The method of 75, wherein the composition has a zero shear viscosity less than 1200 centipoise and greater than 10 centipoise at 25° C.
80. The method of any one of 75 to 79, wherein the insoluble beneficial agent complex comprises protamine.
81. The method of any one of 75 to 80, wherein the composition further comprises an antioxidant.
82. The method of claim 81, wherein the antioxidant is present in an amount ranging from about 1 wt % to about 45 wt %, relative to the amount of beneficial agent.
83. The method of any one of 75 to 80, wherein the composition further comprises methionine.
84. The method of 83, wherein the methionine is present in an amount from about 0.1 wt % to about 45 wt % of the beneficial agent.
85. The method of 84, wherein the methionine is present in an amount from about 1 wt % to about 45 wt % of the beneficial agent.
86. The method of any one of 75 to 85, wherein the insoluble beneficial agent complex comprises a beneficial agent having a molecular weight greater than 5 kD.
87. The method of any one of 75 to 86, wherein the insoluble beneficial agent complex comprises a beneficial agent having a molecular weight greater than 10 kD.
88. The method of any one of 75 to 87, wherein the insoluble beneficial agent complex comprises a divalent metal salt of the beneficial agent.
89. The method of 88, wherein the divalent metal is selected from Zn²⁺, Mg²⁺ and Ca²⁺.
90. The method of 88, wherein the divalent metal is Zn²⁺.
91. The method of any one of 75 to 90, wherein, following administration of the composition, the beneficial agent is present at detectable levels in the plasma of the subject for an extended period of time relative to administration of the beneficial agent alone or administration of the beneficial agent in the hydrophobic solvent alone.
92. The method of any one of 75 to 91, wherein the composition is administered to the subject using a needle of 21 gauge or smaller.
93. The method of any one of 75 to 91, wherein the composition is administered to the subject using a needle of 21 gauge to 27 gauge.
94. The method of any one of 75 to 91, wherein the injectable composition is administered to the subject using a needleless injector.
95. The method of any one of 75 to 94, wherein, following administration of the composition, the mean residence time (MRT) of beneficial agent in-vivo is greater than the sum of MRTₛₒₗᵥₑₙₜ+ΔMRT_{complex}×ΔMRT_{polymer}, wherein MRTₛₒₗᵥₑₙₜ is the MRT for the beneficial agent in the hydrophobic solvent alone, ΔMRT_{complex} is the change in MRT due to the insoluble beneficial agent complex in the absence of polymer, and ΔMRT_{polymer} is the change in MRT due to the polymer in the absence of complexation of the beneficial agent.
96. The method of 95, wherein the MRT of the beneficial agent is up to about 10 fold greater than the sum of MRTₛₒₗᵥₑₙₜ+ΔMRT_{complex}+ΔMRT_{polymer}.
97. A method of making a composition, comprising:
   combining a biodegradable polymer and a hydrophobic solvent to form a single-phase vehicle of the composition,
      wherein the biodegradable polymer is included in an amount of from about 5% to about 40% by weight of the vehicle, and
      the hydrophobic solvent is included in an amount of from about 95% to about 60% by weight of the vehicle;
   dispersing an insoluble component comprising beneficial agent in the vehicle to form the composition; and
   irradiating the composition with ionizing radiation, wherein the beneficial agent maintains a purity of about 90% or greater when stored at 25° C. for a period of 24 hours after irradiation.

### BRIEF DESCRIPTION OF THE DRAWINGS

FIG. 1 provides reverse phase high pressure liquid chromatography (RPLC) spectra for control hGH powder showing stability before gamma irradiation at a dose of 15 kGy (A), control hGH powder after gamma irradiation at a dose of 15 kGy (B), and hGH:protamine complex powder with methionine (at 35 wt % to hGH) after gamma irradiation at a dose of 15 kGy (C). The peak at retention time (Rₜ) around 9.8 min represents pure hGH; whereas peaks at 8.15, 9.3, 10.5 and 11.2 min are impurity peaks. The hGH powder exposed to Gamma-irradiation at a dose of 15 kGy showed an impurity peak around 8.15 min which is not seen in the hGH:protamine complex powder with methionine. The purity of hGH was maintained by complexing with protamine and including methionine as an additive.

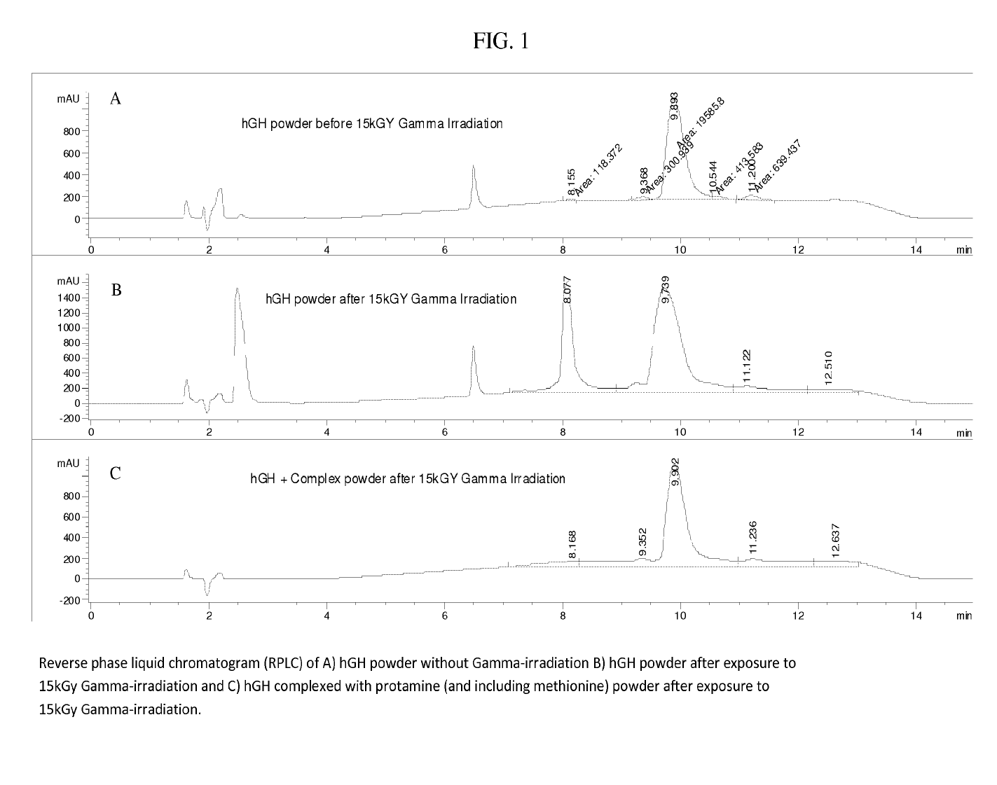

FIG. 2 provides reverse phase high pressure liquid chromatography (RPLC) spectra showing suspension stability for hGH complexed with protamine in a SAM (sucrose acetate isobutyrate)/BB (benzyl benzoate)/PLA (polylactic acid) (8:72:20, % w/w) vehicle prepared as a two-component formulation (non-irradiated) (A), hGH complexed with protamine in a SAIB/BB/PLA (8:72:20, % w/w) vehicle prepared as a two-component formulation (10 kGy-irradiated) (B), and hGH complexed with protamine in a SAIB/BB/PLA (8:72:20, % w/w) vehicle prepared as a two-component formulation (15 kGy-irradiated) (C). The two-component system included complex powder in one syringe and vehicle loaded in another syringe. Both syringes were mixed by hand mixing for about 40 cycles and the resultant suspension was analyzed with and without Gamma-irradiation. The impurity level increased and the purity of hGH (peak about 19 min) decreased with increased exposure to Gamma-irradiation.

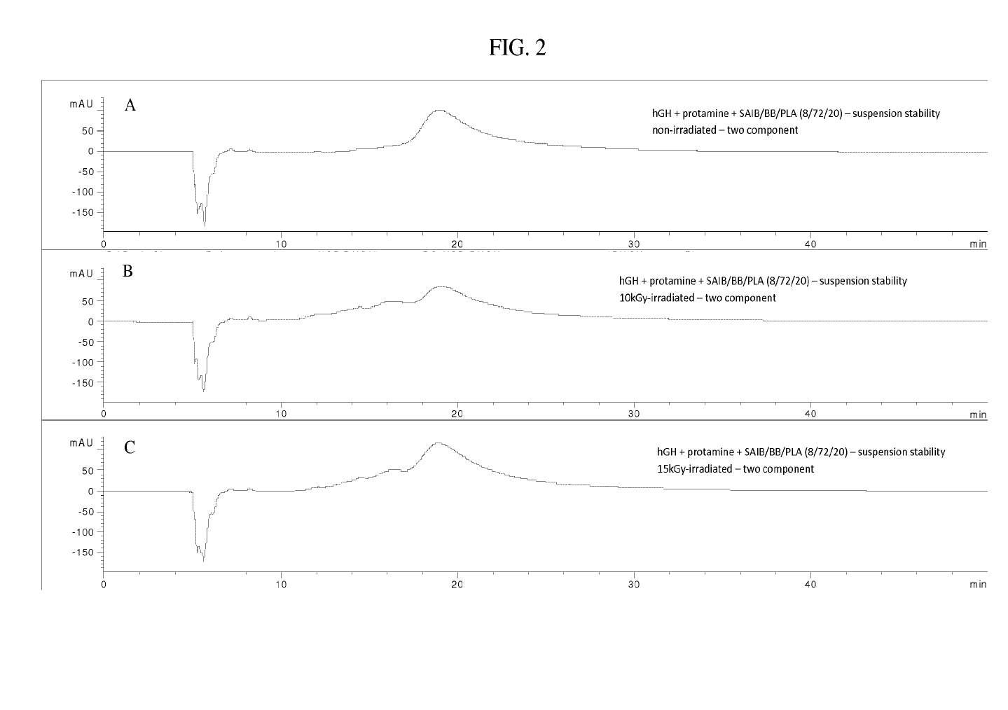

FIG. 3 provides reverse phase high pressure liquid chromatography (RPLC) spectra showing suspension stability for hGH complexed with protamine in a SAIB/BB/PLA (8:72:20, % w/w) vehicle prepared as a one-component formulation (non-irradiated) (A), hGH complexed with protamine in a SAIB/BB/PLA (8:72:20, % w/w) vehicle prepared as a one-component formulation (10 kGy-irradiated) (B), and hGH complexed with protamine in a SAIB/BB/PLA (8:72:20) vehicle prepared as a one-component formulation (15 kGy-irradiated) (C). The one-component system included complex powder in one vial which was mixed with vehicle by homogenization. The resultant suspension was analyzed with and without Gamma-irradiation. The impurity level increased and the purity of hGH (peak about 19 min) decreased with increased exposure to Gamma-irradiation.

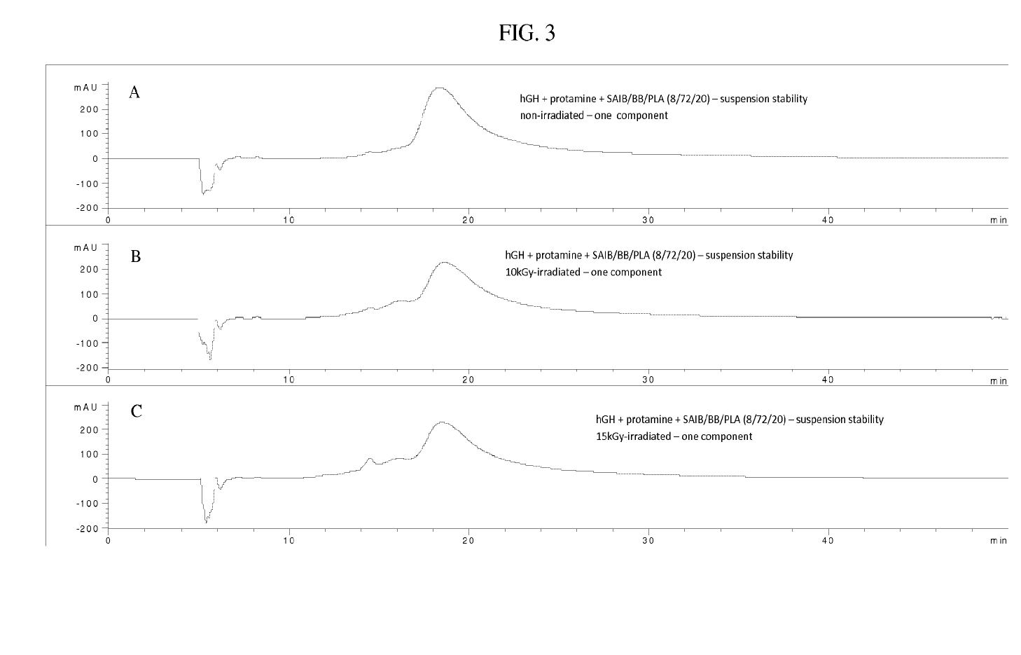

FIG. 4 provides reverse phase high pressure liquid chromatography (RPLC) spectra showing suspension stability for hGH complexed with protamine and including methionine (35 wt % to hGH) as an additive in a SAIB/BB/PLA (8:72:20, % w/w) vehicle prepared as a one-component formulation (2.5-7.5 kGy-irradiated) (A), hGH complexed with protamine and including methionine (35 wt % to hGH) as an additive in a SAIB/BB/PLA (8:72:20, % w/w) vehicle prepared as a one-component formulation (7.5-12.5 kGy-irradiated) (B), hGH complexed with protamine and including methionine (35 wt % to hGH) as an additive in a SAIB/BB/PLA (8:72:20, % w/w) vehicle prepared as a one-component formulation (12.5-17.5 kGy-irradiated) (C), hGH complexed with protamine and including methionine (35 wt % to hGH) as an additive in a SAIB/BB/PLA (8:72:20, % w/w) vehicle prepared as a one-component formulation (22.5-27.5 kGy-irradiated) (D), and a pure hGH powder obtained from USP (Std) (E). The purity of hGH was retained even after exposure of 5-25 kGy Gamma-irradiation. The purity of hGH was good in view of complexation with protamine and the inclusion of methionine in the formulation which prevents degradation as a result of the exposure to Gamma-rays.

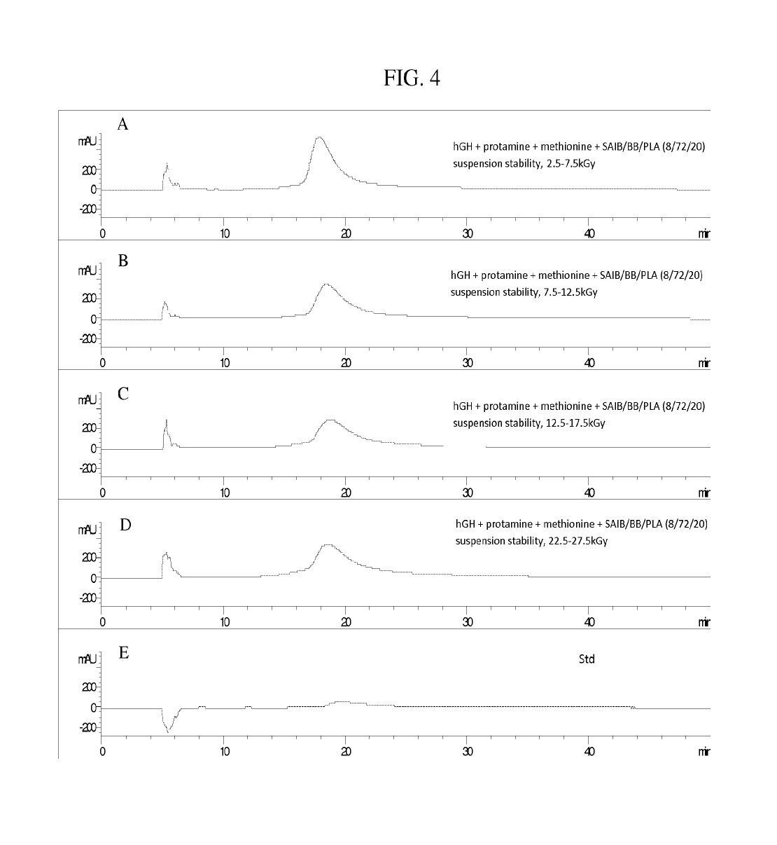

FIG. 5 provides in-vitro release profiles of hGH+protamine complex powder (including methionine) (spray-dried) in SAIB/BB/PLA (8/72/20, % w/w) vehicle with and without exposure to 15 kGy Gamma-radiation. The % cumulative release from the formulation without Gamma-radiation was less than 2% of the total hGH loaded in the formulation (50 mg/mL) up to 10 days and increased significantly to 11% in 15 days. The % cumulative release from the 15 kGy radiated sample showed a similar trend as the formulation without radiation. These release samples were obtained for each time point with 1 mL of fresh Phosphate Buffered Saline (PBS, 10 mM) solution and 1004, of suspension with duplicate samples (n=2).

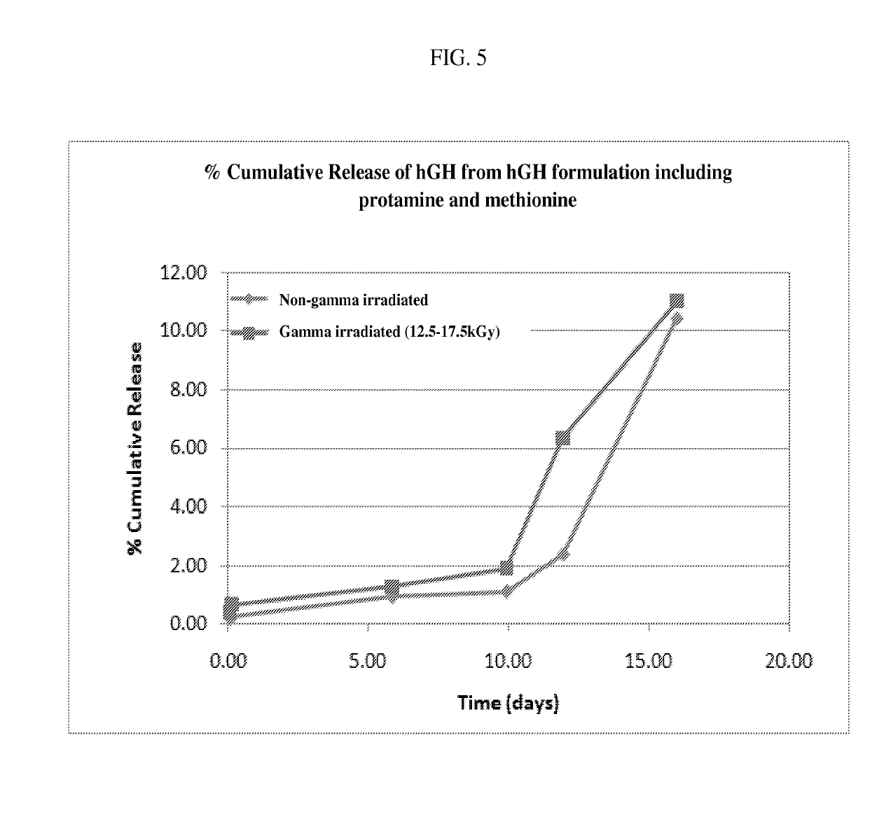

FIG. 6 provides the in-vivo release profiles of hGH+zinc/protamine complex powder (with methionine) (spray-dried) in BB:BA:PLA (70:10:20, % w/w) vehicle with and without exposure of 15 kGy Gamma-radiation. The serum hGH levels from the formulation without Gamma-radiation showed hGH level of 1 ng/mL from the total hGH loaded in the formulation (50 mg/mL) up to 28 days. The serum hGH level from the 15 kGy radiated sample showed the same trend as the formulation without radiation. These serum levels were obtained by hGH ELISA assay from serum samples of 6 rats per formulation at different time points up to 28 days.

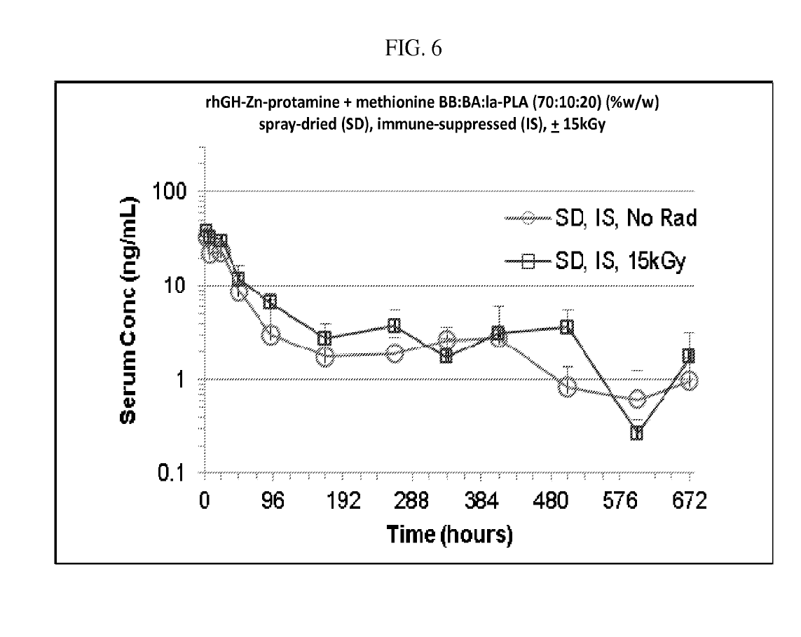

FIG. 7 provides in-vivo release profiles of hGH+zinc/protamine complex powder (with methionine) (lyophilized) in BB:BA:PLA (70:10:20, % w/w) vehicle with and without exposure of 15 kGy Gamma-radiation. The serum hGH levels from the formulation without Gamma-radiation shows hGH levels of 1 ng/mL from the total hGH loaded in the formulation (50 mg/mL) up to 28 days. The serum hGH level resulting from the 15 kGy radiated sample showed the same trend as the formulation without radiation up to 1 week. The pK profiles for gamma-irradiated and non-gamma-irradiated formulations were similar. These serum levels were obtained by hGH ELISA assay from serum samples of 6 rats per formulation at different time points up to 28 days.

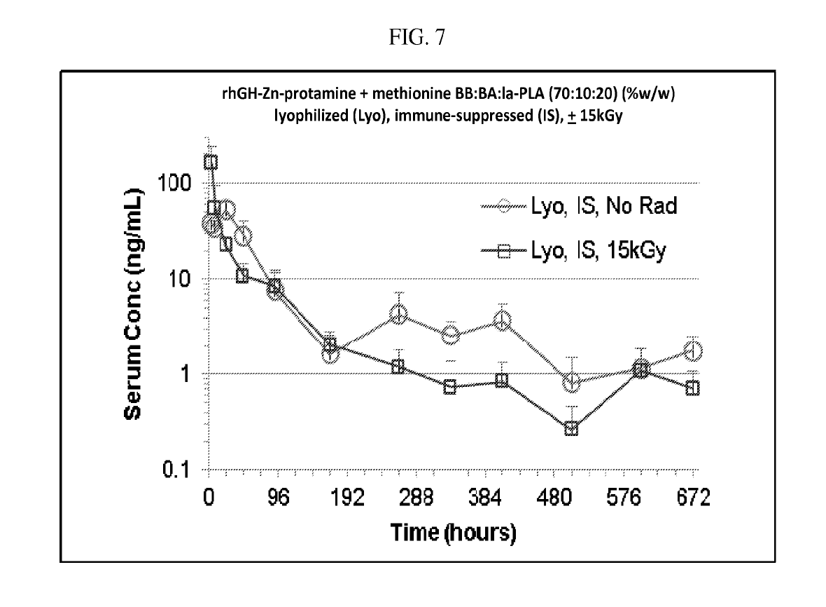

FIG. 8 provides RPLC spectra for complexed and uncomplexed hGH protein formulations. hGH with Sucrose+Methionine was spray-dried and mixed with BB/PLA (80/20) (top); hGH with Zinc+Sucrose+Methionine was spray-dried and mixed with BB/PLA (80/20) (middle); and hGH with Zinc/Protamine+Sucrose+Methionine was spray-dried and mixed with BB/PLA (80/20) (bottom). RPLC was performed on each formulation 24 hours after exposure to a 25 kGy dose of Gamma radiation.

### DEFINITIONS

As used herein, the term “insoluble component” refers to a component of a composition as described herein which includes an insoluble beneficial agent and/or an insoluble beneficial agent complex as defined herein.

As used herein, the term “insoluble beneficial agent” refers to a beneficial agent which is completely or substantially insoluble. The term “substantially insoluble” as used in this context means that at least 90%, e.g., at least 95%, at least 98%, at least 99%, or at least 99.5% of the beneficial agent is insoluble in the vehicle at 25° C. For example, from 90% to 95%, from 95% to 98%, from 98% to 99% or from 99% to 99.5% of the beneficial agent is insoluble in the vehicle at 25° C. For instance, an insoluble beneficial agent is a beneficial agent which may be dispersed in a vehicle and which is not significantly dissolved in the vehicle. An insoluble beneficial agent may include, e.g., a molecule which is substantially insoluble in a vehicle composition as described herein. An insoluble beneficial agent may include, for example, a beneficial agent having a solubility of less than 1 mg/mL in the vehicle at 25° C., e.g., a solubility of from about 0.9 mg/mL to about 0.1 mg/mL, about 0.8 mg/mL to about 0.1 mg/mL, about 0.7 mg/mL to about 0.1 mg/mL, about 0.6 mg/mL to about 0.1 mg/mL, about 0.5 mg/mL to about 0.1 mg/mL, about 0.4 mg/mL to about 0.1 mg/mL, about 0.3 mg/mL to about 0.1 mg/mL, or about 0.2 mg/mL to about 0.1 mg/mL.

As used herein, the term “insoluble beneficial agent complex” refers to beneficial agent complexes which are completely or substantially insoluble in the vehicle. The term “substantially insoluble” as used in this context means that at least 90%, e.g., at least 95%, at least 98%, at least 99%, or at least 99.5% of the beneficial agent complex is insoluble in the vehicle at 25° C. For example, from 90% to 95%, from 95% to 98%, from 98% to 99%, or form 99% to 99.5% of the beneficial agent complex is insoluble in the vehicle at 25° C. For instance, an insoluble beneficial agent complex is a complex which may be dispersed in a vehicle and which is not significantly dissolved in the vehicle. An insoluble beneficial agent complex may include, e.g., a charge-neutralized complex. An insoluble beneficial agent complex may include, for example, a beneficial agent having a solubility of less than 1 mg/mL in the vehicle at 25° C.

The term “charge-neutralized complex” is used herein to refer to a complex formed as a result of a non-covalent charge-based interaction between a beneficial agent and an associated molecule, metal, counter ion, etc., and having no net charge or substantially no net charge. Included within this definition are charge neutralized beneficial agents including salts of the beneficial agents.

As used herein, the term “vehicle” means a composition including a biodegradable polymer and a hydrophobic solvent in the absence of a beneficial agent as described herein.

As used herein, the term “zero shear viscosity” means viscosity at zero shear rate. A skilled artisan would be able to determine zero shear viscosity by measuring viscosity at low shear rate (e.g., around 1 sec⁻¹ to 7 sec⁻¹) using a plate and cone viscometer (e.g., Brookfield Model DV-III+(LV)) and then extrapolating a plot of viscosity versus shear rate to a shear rate of zero at a temperature of interest.

As used herein, the term “emulsion” means a stable mixture of two or more immiscible liquids, including a continuous phase and a dispersed phase.

As used herein, the term “emulsifying agent” means an agent which when included in a biodegradable composition as described herein tends to form an emulsion.

As used herein, the terms “beneficial agent” and “active agent” are interchangeably to mean an agent, e.g., a protein, peptide, nucleic acid (including nucleotides, nucleosides and analogues thereof) or small molecule drug, that provides a desired pharmacological effect upon administration to a subject, e.g., a human or a non-human animal, either alone or in combination with other active or inert components. Included in the above definition are precursors, derivatives, analogues and prodrugs of beneficial agents.

As used herein, the term “non-aqueous” refers to a substance that is substantially free of water. Non-aqueous compositions have a water content of less than about 5%, such as less than about 2%, less than about 1%, less than 0.5%, or less than 0.1%, by weight. For example, non-aqueous compositions may have a water content of from less than 5% to about 0.1%, e.g., from less than 5% to about 2%, from about 2% to about 1%, from about 1% to about 0.5%, or from about 0.5% to about 0.1%. The present compositions are typically non-aqueous.

As used herein, the terms “burst effect” and “burst” are used interchangeably to mean a rapid, initial release of beneficial agent from a composition following administration of the composition which may be distinguished from a subsequent relatively stable, controlled period of release.

As used herein the term “syringeability” describes the ability of a composition to pass easily through a hypodermic needle on transfer from a container prior to injection. Syringeability may be quantified, for example, by measuring the force required to move a known amount of a composition through a syringe and needle, per unit time.

As used herein the term “injectability” refers to the performance of a composition during injection and includes factors such as pressure or force required for injection, evenness of flow, aspiration qualities, and freedom from clogging. Injectability may be quantified e.g., by measuring the force required to move a known amount of a composition through a syringe and needle, per unit time.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid,” “nucleic acid molecule”, “oligonucleotide” and “polynucleotide” are used interchangeably and refer to a polymeric form of nucleotides of any length, either deoxyribonucleotides or ribonucleotides, or compounds produced synthetically which can hybridize with naturally occurring nucleic acids in a sequence specific manner similar to that of two naturally occurring nucleic acids, e.g., can participate in Watson-Crick base pairing interactions. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, cDNA, recombinant polynucleotides, plasmids, vectors, isolated DNA of any sequence, control regions, isolated RNA of any sequence, nucleic acid probes, and primers.

The terms “rate controlling cloud,” “rate controlling film,” and “rate controlling surface layer” are used interchangeably herein to refer to a rate controlling element of a formulation which is formed at the formulation surface and an aqueous environment, which surrounds a substantially liquid core and has a release rate-controlling effect on a beneficial agent from the substantially liquid core of the formulation to the aqueous environment. Unlike polymeric matrices that are formed by a phase inversion, phase separation, or gelation process in an aqueous environment, the rate controlling cloud or film does not have appreciable physical strength or mechanical structure.

As used herein “bioavailability” refers to the fraction of the beneficial agent dose that enters the systemic circulation following administration.

As used herein “mean residence time (MRT)” refers to the average total time molecules of a given dose reside in the body which may be calculated as area under the first moment curve (AUMC)/area under the curve (AUC), where

AUC=∫₀^{∞}Cₚ(t)dt

and

AUMC=∫₀²⁸Cₚ(t)·tdt

and, where Cₚ(t) is plasma (or serum or blood) concentration as a function of time.

As used herein, the term “gel” refers to a composition which has a relatively small G″/G′ ratio, for example less than or equal to one, wherein G″=the loss modulus and G′=the storage modulus. Conversely, the terms “non-gel”, “not a gel” and the like refer to a composition which has a relatively large G″/G′ ratio, e.g., a G″/G′ ratio of greater than or equal to 10.

As used herein, the terms “gelling”, “gel-forming” and the like refer to a composition which has a relatively small G″/G′ ratio, for example less than or equal to one (e.g., following aging at 37° C. for a period of 14 days), wherein G″=the loss modulus and G′=the storage modulus. Conversely, the terms “non-gelling”, “non-gel forming” and the like are used herein to refer to a composition which has a relatively large G″/G′ ratio, e.g., a G″/G′ ratio of greater than or equal to 10 (e.g., following aging at 37° C. for a period of 14 days).

As used herein “physical stability” refers to the ability of a material, e.g., a compound or complex to resist physical change.

As used herein “chemical stability” refers to the ability of a material, e.g., a compound or complex to resist chemical change.

As used herein, the terms “Glucagon-like-peptide-1” and “GLP-1” refer to a molecule having GLP-1 activity. One of ordinary skill in the art can determine whether any given moiety has GLP-1 activity, as disclosed in U.S. Published Application No. 2010/0210505, which is incorporated herein by reference. The term “GLP-1” includes native GLP-1 (GLP-1 (7-37)OH or GLP-1 (7-36)NH₂), GLP-1 analogs, GLP-1 derivatives, GLP-1 biologically active fragments, extended GLP-1 (see, for example, International Patent Publication No. WO 03/058203, which is incorporated herein by reference, in particular with respect to the extended glucagon-like peptide-1 analogs described therein), exendin-4, exendin-4 analogs, and exendin-4 derivatives comprising one or two cysteine residues at particular positions as described in WO 2004/093823, which is incorporated herein by reference.

As used herein, “sterile” refers to the composition meeting the requirements of sterility enforced by medicine regulatory authorities, such as the MCA in the UK or the FDA in the US. Tests are included in current versions of the compendia, such as the British Pharmacopoeia and the US Pharmacopoeia. For instance, the composition in question may be essentially free of viable micro-organisms according to Ph. Eur. 2.6. For example, the terminally sterilized composition may have a probability of nonsterility (PNS) of not more than one in a million units produced. This is often stated as a PNS of 10⁻⁶, or the probablility of product bioburden surviving the sterilization process in any single unit of product is less than one in one million.

When used to characterize a vehicle component or components as described herein, the term “% w/w” refers to % by weight of the vehicle, for example, SAM/BB/PLA (8:72:20, % w/w) identifies a vehicle including SAM at 8% by weight of the vehicle, BB at 72% by weight of the vehicle, and PLA at 20% by weight of the vehicle.

Before the present invention is further described, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an insoluble beneficial agent complex” includes a plurality of such complexes and reference to “the injectable depot composition” includes reference to one or more injectable depot compositions and equivalents thereof, and so forth. It is further noted that the claims may be drafted to exclude any optional element. As such, this statement is intended to provide antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As discussed above, the present disclosure is directed to a method of making a composition by combining a vehicle, e.g., a single phase vehicle, and an insoluble component comprising a beneficial agent, and sterilizing the composition prior to use using ionizing radiation, wherein the beneficial agent is radiation stable following sterilization with the ionizing radiation. Related compositions and methods are also provided.

Biocompatible-Biodegradable Polymers

A variety of polymers may be suitable for use in the compositions of the present disclosure provided that they are both biocompatible and biodegradable. For example, suitable polymers may include, but are not limited to, homopolymers, block-copolymers and random copolymers. The polymers may be linear or branched. Suitable polymers include those polymers or combinations of polymers which have solubility of at least about 20 weight %, 30 weight %, or 40 weight % in the selected solvent or solvent combination, e.g., from about 20 weight % to about 30 weight %, or from about 30 weight % to about 40 weight %. In some embodiments, suitable polymers include polymers having both hydrophilic and hydrophobic regions, e.g., an AB-type block copolymer composed of hydrophobic and hydrophilic components. Such polymers may have a tendency to form micelles when exposed to an aqueous environment as a result of the amphiphilic character of the polymer. Suitable polymers may include, but are not limited to, polylactides, polyglycolides, polycaprolactones, copolymers including any combination of two or more monomers involved in the above, e.g., lactide-caprolactone copolymers, glycolide-caprolactone copolymers, terpolymers of lactide, glycolide and ε-caprolactone, and mixtures including any combination of two or more of the above. In other words, suitable polymers may also include, for example, polylactic acids, polyglycolic acids, polycaprolactones, copolymers including any combination of two or more monomers involved in the above, e.g., terpolymers of lactic acid, glycolic acid and ε-caprolactone, and mixtures including any combination of two or more of the above.

In some embodiments, the biodegradable polymer is polylactic acid (PLA), e.g., a PLA including an ionizable end-group (e.g., an acid end-group, e.g., in an acid-terminated PLA). Acid end-group PLAs include, e.g., lactate initiated PLAs described herein. In some embodiments, the PLA includes an unionizable end-group (e.g., an ester end-group, e.g., in an ester terminated PLA). Ester end-group PLAs include, but are not limited to, dodecanol-initiated (dd) PLAs described herein. In some embodiments, the PLA is dl-PLA. In other embodiments, the biodegradable polymer is poly(lactic-co-glycolic acid) (PLGA), e.g., dl-PLGA. In some embodiments, the PLGA includes an ionizable end-group, e.g., an acid end-group. Acid end-group PLGAs include, but are not limited to, the glycolate initiated (ga) PLGAs described herein. In some embodiments, the PLGA includes an unionizable end-group, e.g., an ester end group. Ester end-group PLGAs include, but are not limited to, dodecanol initiated PLGAs described herein. In one embodiment, where the polymer is a polycaprolactone, the polycaprolactone is poly(ε)caprolactone. In some embodiments, a suitable initiator for a biocompatible, biodegradable polymer is glycolic acid, lactic acid or any other suitable acid.

The biocompatible, biodegradable polymer is present in the vehicle in an amount ranging from about 5% to about 40% by weight of the vehicle, for example, from about 6% to about 35%, from about 7% to about 30%, from about 8% to about 27%, from about 9% to about 26%, from about 10% to about 25%, from about 11% to about 24%, from about 12% to about 23%, from about 13% to about 22%, from about 14% to about 21%, from about 15% to about 20%, from about 16% to about 19%, or at about 17% by weight of the vehicle. In some embodiments, the polymer is present in an amount of about 20% by weight of the vehicle.

In some embodiments, the biocompatible, biodegradable polymer has a weight average molecular weight of from about 2 kD to about 20 kD, e.g., from about 2 kD to about 5 kD, from about 2 kD to about 10 kD, or from about 2 kD to about 15 kD. Additional embodiments include a biocompatible, biodegradable polymer having a weight average molecular weight of from about 5 kD to about 15 kD, e.g., about 10 kD.

In some embodiments, where the biocompatible, biodegradable polymer is a PLA or a PLGA, the polymer has an L:G ratio of from 100:0 to 50:50, e.g., the lactide component may range from 100% to 50%, e.g., from 90% to 50%, from 80% to 50%, from 70% to 50%, or from 60% to 50%, while the glycolide component ranges from 0% to 50%, e.g., from 10% to 50%, from 20% to 50%, from 30% to 50%, or from 40% to 50% of the PLA or PLGA. In some embodiments, where the biocompatible, biodegradable polymer is a PLGA, the polymer has an L:G ratio of about 95:5, about 90:10, about 85:15, about 80:20, about 75:25, about 70:30, about 65:35, about 60:40, or about 55:45.

Solvents

Hydrophobic solvents suitable for use in the compositions of the present disclosure are hydrophobic solvents which are capable of solubilizing a polymer component of the vehicles described herein. Hydrophobic solvents can be characterized as being insoluble or substantially insoluble in water. For example, suitable hydrophobic solvents have solubility in water of less than 5% by weight, less than 4% by weight, less than 3% by weight, less than 2% by weight or less than 1% by weight, e.g. as measured at 25° C. A suitable hydrophobic solvent may also be characterized as one which has a solubility in water of about 5% or less, about 4% or less, about 3% or less, about 2% or less, or about 1% or less, at 25° C. For example, in some embodiments, a suitable hydrophobic solvent has a solubility in water of from about 1% to about 5%, from about 1% to about 4%, from about 1% to about 3%, and from about 1% to about 2%, at 25° C. A suitable hydrophobic solvent may also be characterized as a solvent in which water has limited solubility, e.g., a solvent in which water has solubility of less than 10% by weight, less than 5% by weight, or less than 1% by weight, at 25° C., e.g., from less 10% by weight to about 1% by weight, or from less than 5% by weight to about 1% by weight. In some embodiments, a suitable hydrophobic solvent is one which solubilizes the polymer component of the vehicle and which when combined with the polymer component in a suitable amount as described herein results in a vehicle having a low viscosity, i.e., a zero shear viscosity less than 1,200 centipoise at 25° C.

Hydrophobic solvents find particular use in the preparation of radiation stable formulations because such solvents allow less transmission of Gamma radiation than hydrophilic solvents.

In some embodiments, suitable solvents include derivatives of benzoic acid including, but not limited to, benzyl alcohol, methyl benzoate, ethyl benzoate, n-propyl benzoate, isopropyl benzoate, butyl benzoate, isobutyl benzoate, sec-butyl benzoate, tert-butyl benzoate, isoamyl benzoate and benzyl benzoate. In some embodiments, benzyl benzoate is selected as the hydrophobic solvent for use in the biodegradable delivery compositions of the present disclosure.

A suitable solvent may be a single solvent selected from among the following or a combination of two or more of the following: benzyl alcohol, benzyl benzoate, ethyl benzoate, and ethanol (EtOH).

Where the solvent is a hydrophobic solvent, it may be used in combination with one or more additional solvents, e.g., one or more hydrophobic solvents and/or one or more polar/hydrophilic solvents.

In some embodiments, the compositions include a single hydrophobic solvent as described herein without including any additional solvents. In some embodiments, the single hydrophobic solvent is benzyl benzoate, in other embodiments the single hydrophobic solvent is other than benzyl alcohol.

Where the solvent is a polar/hydrophilic solvent, it is used in the disclosed compositions only in combination with a hydrophobic solvent and is present in a relatively small amount relative to the hydrophobic solvent, e.g., less than 5% (e.g., less than 4%, less than 3%, less than 2%, or less than 1%) by weight of the vehicle. For example, a polar/hydrophilic solvent may be present in the vehicle in an amount of from about 5% to about 1% (e.g., from about 4% to about 1%, from about 3% to about 1%, or from about 2% to about 1%) by weight of the vehicle. Without wishing to be bound by any particular theory, it is believed that the addition of relatively small amounts of polar/hydrophilic solvent, e.g., ethanol, to the vehicle composition may broaden the range of polymers in terms of polymer type, molecular weight, and relative hydrophobicity/hydrophilicity which may be utilized in the disclosed compositions.

Suitable polar/hydrophilic solvents which may be used in combination with a hydrophobic solvent as described herein may include, e.g., ethanol, methanol, n-propanol, dimethyl sulfoxide (DMSO), and N-Methyl-2-pyrrolidone (NMP).

The hydrophobic solvent (or combination of hydrophobic solvents) is present in the vehicle from about 95% to about 60% by weight of the vehicle, for example, from about 94% to about 61%, from about 93% to about 62%, from about 92% to about 63%, from about 91% to about 64%, from about 90% to about 65%, from about 89% to about 66%, from about 88% to about 67%, from about 87% to about 68%, from about 86% to about 69%, from about 85% to about 70%, from about 84% to about 71%, from about 83% to about 72%, from about 82% to about 73%, from about 81% to about 74%, from about 80% to about 75%, from about 79% to about 76%, or from about 78% to about 77% by weight of the vehicle. In some embodiments, the hydrophobic solvent (or combination of hydrophobic solvents) is present in the vehicle from about 95% to about 90%, from about 95% to about 85%, from about 95% to about 80%, from about 95% to about 75%, from about 95% to about 70%, from about 95% to about 65%, or from about 95% to about 60% by weight of the vehicle. In some embodiments, the hydrophobic solvent is present in an amount of about 80% by weight of the vehicle. In other embodiments, the hydrophobic solvent is present in an amount of about 72% by weight of the vehicle.

In some embodiments, the biodegradable drug delivery compositions disclosed herein are free of hydrophilic solvent. In some embodiments, the biodegradable delivery compositions disclosed herein do not include a thixotropic agent, e.g., a lower alkanol containing 2-6 carbon atoms.

Beneficial Agents

A variety of beneficial agents may be delivered using the biodegradable delivery compositions disclosed herein. General classes of beneficial agents which may be delivered include, for example, proteins, peptides, nucleic acids, nucleotides, nucleosides and analogues thereof, antigens, antibodies, and vaccines; as well as low molecular weight compounds.

In some embodiments, the beneficial agent is at least substantially insoluble in the vehicle, e.g., solubility in the vehicle less than 10 mg/mL, less than 5 mg/mL, less than 1 mg/mL, less than 0.5 mg/mL, less than 0.3 mg/mL, less than 0.2 mg/mL, or less than 0.1 mg/mL. For example, the beneficial agent may have a solubility in the vehicle of less than 10 mg/mL to about 0.1 mg/mL, less than 5 mg/mL to about 0.1 mg/mL, less than 1 mg/mL to about 0.1 mg/mL, less than 0.9 mg/mL to about 0.1 mg/mL, less than 0.8 mg/mL to about 0.1 mg/mL, less than 0.7 mg/mL to about 0.1 mg/mL, less than 0.6 mg/mL to about 0.1 mg/mL, less than 0.5 mg/mL to about 0.1 mg/mL, less than 0.4 mg/mL to about 0.1 mg/mL, less than 0.3 mg/mL to about 0.1 mg/mL, or about 0.2 mg/mL to about 0.1 mg/mL.

In some embodiments, the beneficial agent has a molecular weight from about 200 D to about 1000 kD, e.g., from about 10 kD to about 150 kD, from about 20 kD to about 100 kD, from about 30 kD to about 80 kD, from about 40 kD to about 70 kD, or from about 50 kD to about 60 kD. Beneficial agents which may be delivered using the biodegradable delivery compositions disclosed herein include, but are not limited to, agents which act on the peripheral nerves, adrenergic receptors, cholinergic receptors, the skeletal muscles, the cardiovascular system, smooth muscles, the blood circulatory system, synaptic sites, neuroeffector junction sites, endocrine and hormone systems, the immunological system, the reproductive system, the skeletal system, autacoid systems, the alimentary and excretory systems, the histamine system and the central nervous system.

Suitable beneficial agents may be selected, for example, from chemotherapeutic agents, epigenetic agents, proteasome inhibitors, adjuvant drugs, anti-emetics, appetite stimulants, anti-wasting agents and high potency opioids.

Suitable beneficial agents may also be selected, for example, from anti-neoplastic agents, cardiovascular agents, renal agents, gastrointestinal agents, rheumatologic agents and neurological agents among others.

Protein, Polypeptides and Peptides as Beneficial Agents

Proteins useful in the disclosed formulations may include, for example, molecules such as cytokines and their receptors, as well as chimeric proteins comprising cytokines or their receptors, including, for example tumor necrosis factor alpha and beta, their receptors and their derivatives; renin; growth hormones, including human growth hormone, bovine growth hormone, methione-human growth hormone, des-phenylalanine human growth hormone, and porcine growth hormone; growth hormone releasing factor (GRF); parathyroid and pituitary hormones; thyroid stimulating hormone; human pancreas hormone releasing factor; lipoproteins; colchicine; prolactin; corticotrophin; thyrotropic hormone; oxytocin; vasopressin; somatostatin; somatostatin analogs; octreotide; lypressin; pancreozymin; leuprolide; alpha-1-antitrypsin; insulin A-chain; insulin B-chain; proinsulin; follicle stimulating hormone; calcitonin; luteinizing hormone; luteinizing hormone releasing hormone (LHRH); LHRH agonists and antagonists; glucagon; clotting factors such as factor VIIIC, factor IX, tissue factor, and von Willebrands factor; anti-clotting factors such as Protein C; atrial natriuretic factor; lung surfactant; a plasminogen activator other than a tissue-type plasminogen activator (t-PA), for example a urokinase; bombesin; thrombin; hemopoietic growth factor; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; gonadotropin releasing hormone; bovine somatotropin; porcine somatotropin; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as acidic FGF and basic FGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha (e.g., interferonα2A or interferonα2B), -beta, -gamma, -lambda and consensus interferon; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV-1 envelope glycoprotein, gp120, gp160 or fragments thereof; transport proteins; homing receptors; addressins; fertility inhibitors such as the prostaglandins; fertility promoters; regulatory proteins; antibodies and chimeric proteins, such as immunoadhesins; precursors, derivatives, prodrugs and analogues of these compounds, and pharmaceutically acceptable salts of these compounds, or their precursors, derivatives, prodrugs and analogues.

Suitable proteins or peptides may be native or recombinant and include, e.g., fusion proteins.

In some embodiments, the protein is a growth hormone, such as human growth hormone (hGH), recombinant human growth hormone (rhGH), bovine growth hormone, methione-human growth hormone, des-phenylalanine human growth hormone, and porcine growth hormone; insulin, insulin A-chain, insulin B-chain, and proinsulin; or a growth factor, such as vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

Suitable peptides for use as the beneficial agent in the biodegradable delivery compositions disclosed herein include, but are not limited to, Glucagon-like peptide-1 (GLP-1) and precursors, derivatives, prodrugs and analogues thereof. In some embodiments, a suitable peptide is a GLP-1 receptor agonist, e.g., exenatide or liraglutide.

In addition, a suitable protein, polypeptide, peptide; or precursor, derivative, prodrug or analogue thereof is one which is capable of forming an insoluble component comprising beneficial agent, e.g., an insoluble beneficial agent complex, e.g., by complexing with a metal or other precipitating and/or stabilizing agent as described herein.

In some embodiments, the beneficial agent comprises growth hormone and the hydrophobic solvent does not comprise benzyl alcohol. In some embodiments, the beneficial agent comprises growth hormone and the hydrophobic solvent does not comprise ethyl benzoate.

In some embodiments, the beneficial agent comprises a peptide having a molecular weight of from about 1000 Daltons to about 5000 Daltons, e.g., from about 2000 Daltons to about 5000 Daltons, from about 3000 Daltons to about 5000 Daltons, or from about 4000 Daltons to about 5000 Daltons. In some embodiments, the beneficial agent comprises a polypeptide having a molecular weight of from about 4000 Daltons to about 150,000 Daltons, e.g., from about 5 kD to about 150 kD, from about 10 kD to about 150 kD, from about 50 kD to about 150 kD, or from about 100 kD to about 150 kD. In some embodiments, the beneficial agent comprises a large polypeptide having a molecular weight of from about 150,000 Daltons to about 1,000,000 Daltons, e.g., from about 200 kD to about 1000 kD or from about 500 kD to about 1000 kD.

Nucleic Acids as Beneficial Agents

Nucleic acid beneficial agents include nucleic acids as well as precursors, derivatives, prodrugs and analogues thereof, e.g., therapeutic nucleotides, nucleosides and analogues thereof; therapeutic oligonucleotides; and therapeutic polynucleotides. Beneficial agents selected from this group may find particular use as anticancer agents and antivirals. Suitable nucleic acid beneficial agents may include for example ribozymes, antisense oligodeoxynucleotides, aptamers and siRNA. Examples of suitable nucleoside analogues include, but are not limited to, cytarabine (araCTP), gemcitabine (dFdCTP), and floxuridine (FdUTP).

Other Beneficial Agent Compounds

A variety of other beneficial agent compounds may be used in the compositions disclosed herein. Suitable compounds may include, but are not limited to, compounds directed to one or more of the following drug targets: Kringle domain, Carboxypeptidase, Carboxylic ester hydrolases, Glycosylases, Rhodopsin-like dopamine receptors, Rhodopsin-like adrenoceptors, Rhodopsin-like histamine receptors, Rhodopsin-like serotonin receptors, Rhodopsin-like short peptide receptors, Rhodopsin-like acetylcholine receptors, Rhodopsin-like nucleotide-like receptors, Rhodopsin-like lipid-like ligand receptors, Rhodopsin-like melatonin receptors, Metalloprotease, Transporter ATPase, Carboxylic ester hydrolases, Peroxidase, Lipoxygenase, DOPA decarboxylase, A/G cyclase, Methyltransferases, Sulphonylurea receptors, other transporters (e.g., Dopamine transporter, GABA transporter 1, Norepinephrine transporter, Potassium-transporting ATPase α-chain 1, Sodium-(potassium)-chloride cotransporter 2, Serotonin transporter, Synaptic vesicular amine transporter, and Thiazide-sensitive sodium-chloride cotransporter), Electrochemical nucleoside transporter, Voltage-gated ion channels, GABA receptors (Cys-Loop), Acetylcholine receptors (Cys-Loop), NMDA receptors, 5-HT3 receptors (Cys-Loop), Ligand-gated ion channels Glu: kainite, AMPA Glu receptors, Acid-sensing ion channels aldosterone, Ryanodine receptors, Vitamin K epoxide reductase, MetGluR-like GABA_{B} receptors, Inwardly rectifying K⁺ channel, NPC1L1, MetGluR-like calcium-sensing receptors, Aldehyde dehydrogenases, Tyrosine 3-hydroxylase, Aldose reductase, Xanthine dehydrogenase, Ribonucleoside reductase, Dihydrofolate reductase, IMP dehydrogenase, Thioredoxin reductase, Dioxygenase, Inositol monophosphatase, Phosphodiesterases, Adenosine deaminase, Peptidylprolyl isomerases, Thymidylate synthase, Aminotransferases, Farnesyl diphosphate synthase, Protein kinases, Carbonic anhydrase, Tubulins, Troponin, Inhibitor of IκB kinase-β, Amine oxidases, Cyclooxygenases, Cytochrome P450s, Thyroxine 5-deiodinase, Steroid dehydrogenase, HMG-CoA reductase, Steroid reductases, Dihydroorotate oxidase, Epoxide hydrolase, Transporter ATPase, Translocator, Glycosyltransferases, Nuclear receptors NR3 receptors, Nuclear receptors: NR1 receptors, and Topoisomerase.

In some embodiments, the beneficial agent is a compound targeting one of rhodopsin-like GPCRs, nuclear receptors, ligand-gated ion channels, voltage-gated ion channels, penicillin-binding protein, myeloperoxidase-like, sodium: neurotransmitter symporter family, type II DNA topoisomerase, fibronectin type III, and cytochrome P450.

In some embodiments, the beneficial agent is an anticancer agent. Suitable anticancer agents include, but are not limited to, Actinomycin D, Alemtuzumab, Allopurinol sodium, Amifostine, Amsacrine, Anastrozole, Ara-CMP, Asparaginase, Azacytadine, Bendamustine, Bevacizumab, Bicalutimide, Bleomycin (e.g., Bleomycin A₂ and B₂), Bortezomib, Busulfan, Camptothecin sodium salt, Capecitabine, Carboplatin, Carmustine, Cetuximab, Chlorambucil, Cisplatin, Cladribine, Clofarabine, Cyclophosphamide, Cytarabine, Dacarbazine, Dactinomycin, Daunorubicin, Daunorubicin liposomal, Dacarbazine, Decitabine, Docetaxel, Doxorubicin, Doxorubicin liposomal, Epirubicin, Estramustine, Etoposide, Etoposide phosphate, Exemestane, Floxuridine, Fludarabine, Fluadarabine phosphate, 5-Fluorouracil, Fotemustine, Fulvestrant, Gemcitabine, Goserelin, Hexamethylmelamine, Hydroxyurea, Idarubicin, Ifosfamide, Imatinib, Irinotecan, Ixabepilone, Lapatinib, Letrozole, Leuprolide acetate, Lomustine, Mechlorethamine, Melphalan, 6-Mercaptopurine, Methotrexate, Mithramycin, Mitomycin C, Mitotane, Mitoxantrone, Nimustine, Ofatumumab, Oxaliplatin, Paclitaxel, Panitumumab, Pegaspargase, Pemetrexed, Pentostatin, Pertuzumab, Picoplatin, Pipobroman, Plerixafor, Procarbazine, Raltitrexed, Rituximab, Streptozocin, Temozolomide, Teniposide, 6-Thioguanine, Thiotepa, Topotecan, Trastuzumab, Treosulfan, Triethylenemelamine, Trimetrexate, Uracil Nitrogen Mustard, Valrubicin, Vinblastine, Vincristine, Vindesine, Vinorelbine, and analogues, precursors, derivatives and pro-drugs thereof. It should be noted that two or more of the above compounds may be used in combination in the compositions of the present disclosure.

Beneficial agents of interest for use in the disclosed compositions may also include opioids and derivatives thereof as well as opioid receptor agonists and antagonists, e.g., methadone, naltrexone, naloxone, nalbuphine, fentanyl, sufentanil, oxycodone, oxymorphone, hydrocodone, hydromorphone, and pharmaceutically acceptable salts and derivatives thereof.

In some embodiments the beneficial agent is a low molecular weight compound, e.g., a compound having a molecular weight of less than or equal to about 2000 Daltons, e.g., less than or equal to about 1500 Daltons, less than or equal to about 1000 Daltons, less than or equal to about 900 Daltons, less than or equal to about 800 Daltons, less than or equal to about 700 Daltons, less than or equal to about 600 Daltons, less than or equal to about 500 Daltons, less than or equal to about 400 Daltons, less than or equal to about 300 Daltons, less than or equal to about 200 Daltons, less than or equal to about 100 Daltons. For example, a low molecular weight compound may have a molecular weight of from about 2000 Daltons to about 100 Daltons, e.g., from about 1500 Daltons to about 100 Daltons, from about 1000 Daltons to about 100 Daltons, from about 900 Daltons to about 100 Daltons, from about 800 Daltons to about 100 Daltons, from about 700 Daltons to about 100 Daltons, from about 600 Daltons to about 100 Daltons, from about 500 Daltons to about 100 Daltons, from about 400 Daltons to about 100 Daltons, from about 300 Daltons to about 100 Daltons, or from about 200 Daltons to about 100 Daltons. In some embodiments, where the beneficial agent is a low molecular weight compound, the beneficial agent is one which has solubility in water of 10 to 100 mg/ml or less, e.g., less than 100 mg/ml, less than 90 mg/ml, less than 80 mg/ml, less than 70 mg/ml, less than 60 mg/ml, less than 50 mg/ml, less than 40 mg/ml, less than 30 mg/ml, less than 20 mg/ml, less than 10 mg/ml, less than 5 mg/ml, or less than 1 mg/ml. For example, in some embodiments the beneficial agent is a low molecular weight compound which has solubility in water of less than 100 mg/ml to about 1 mg/ml, e.g., less than 90 mg/ml to about 1 mg/ml, less than 80 mg/ml to about 1 mg/ml, less than 70 mg/ml to about 1 mg/ml, less than 60 mg/ml to about 1 mg/ml, less than 50 mg/ml to about 1 mg/ml, less than 40 mg/ml to about 1 mg/ml, less than 30 mg/ml to about 1 mg/ml, less than 20 mg/ml to about 1 mg/ml, less than 10 mg/ml to about 1 mg/ml, or less than 5 mg/ml to about 1 mg/ml.

In some embodiments, a low molecular weight compound suitable for use as a beneficial agent is a compound that is at least substantially insoluble in the vehicle, e.g., solubility in the vehicle is less than 10 mg/mL, less than 5 mg/mL, less than 1 mg/mL, less than 0.5 mg/mL, less than 0.3 mg/mL, less than 0.2 mg/mL, or less than 0.1 mg/mL. For example, the low molecular weight compound may have a solubility in the vehicle of less than 10 mg/mL to about 0.1 mg/mL, less than 5 mg/mL to about 0.1 mg/mL, less than 1 mg/mL to about 0.1 mg/mL, less than 0.9 mg/mL to about 0.1 mg/mL, less than 0.8 mg/mL to about 0.1 mg/mL, less than 0.7 mg/mL to about 0.1 mg/mL, less than 0.6 mg/mL to about 0.1 mg/mL, less than 0.5 mg/mL to about 0.1 mg/mL, less than 0.4 mg/mL to about 0.1 mg/mL, less than 0.3 mg/mL to about 0.1 mg/mL, or about 0.2 mg/mL to about 0.1 mg/mL.

In some embodiments, a low molecular weight compound suitable for use as a beneficial agent is a compound which when present in salt form is at least substantially insoluble in the vehicle, e.g., solubility in the vehicle is less than 10 mg/mL, less than 5 mg/mL, less than 1 mg/mL, less than 0.5 mg/mL, less than 0.3 mg/mL, less than 0.2 mg/mL, or less than 0.1 mg/mL. For example, the low molecular weight compound may have a solubility in the vehicle of less than 10 mg/mL to about 0.1 mg/mL, less than 5 mg/mL to about 0.1 mg/mL, less than 1 mg/mL to about 0.1 mg/mL, less than 0.9 mg/mL to about 0.1 mg/mL, less than 0.8 mg/mL to about 0.1 mg/mL, less than 0.7 mg/mL to about 0.1 mg/mL, less than 0.6 mg/mL to about 0.1 mg/mL, less than 0.5 mg/mL to about 0.1 mg/mL, less than 0.4 mg/mL to about 0.1 mg/mL, less than 0.3 mg/mL to about 0.1 mg/mL, or about 0.2 mg/mL to about 0.1 mg/mL when present in salt form.

The beneficial agent or beneficial agent complex may be present in any suitable concentration in the biodegradable compositions disclosed herein. Suitable concentrations may vary depending on the potency of the beneficial agent, beneficial agent pharmacokinetic half-life, etc. For example, the insoluble component comprising beneficial agent, e.g., insoluble beneficial agent complex, may be present in a range of from about 1% to about 50% by weight of the composition, e.g., from about 5% to about 45%, from about 10% to about 40%, from about 15% to about 35%, or from about 20% to about 30% by weight of the composition. The insoluble component comprising beneficial agent, e.g., insoluble beneficial agent complex, may be present at a concentration ranging from about 10 mg/mL to about 500 mg/mL, such as from about 50 mg/mL to about 450 mg/mL, about 100 mg/mL to about 400 mg/mL, about 150 mg/mL to about 350 mg/mL, or about 200 mg/mL to about 300 mg/mL.

In some embodiments, the beneficial agent is an insoluble beneficial agent as defined herein, i.e., a beneficial agent which is completely or substantially insoluble in the vehicle chosen for use in connection with the biodegradable drug delivery compositions described herein. In other words, at least 90%, e.g., at least 95%, at least 98%, at least 99%, or at least 99.5% of the beneficial agent is insoluble in the vehicle at 25° C. For example, from 90% to 95%, from 95% to 98%, from 98% to 99% or from 99% to 99.5% of the beneficial agent is insoluble in the vehicle at 25° C. An insoluble beneficial agent is a beneficial agent which may be dispersed in a vehicle and which is not significantly dissolved in the vehicle. An insoluble beneficial agent may include, e.g., a molecule which is substantially insoluble in a vehicle composition as described herein.

Insoluble Complex

The beneficial agent may be provided as an insoluble beneficial agent complex, e.g., an electrostatic complex, which is dispersed in the vehicle. Complexing may be used to reduce the solubility of beneficial agents. As defined previously herein, the term “insoluble beneficial agent complex”, includes beneficial agent complexes which are completely or substantially insoluble in the vehicle chosen for use in connection with the biodegradable drug delivery compositions described herein. The term “substantially insoluble” as used in this context means that at least 90%, e.g., at least 95%, at least 98%, at least 99%, or at least 99.5% of the beneficial agent complex is insoluble in the vehicle at 25° C. For example, from 90% to 95%, from 95% to 98%, from 98% to 99% or from 99% to 99.5% of the beneficial agent complex is insoluble in the vehicle at 25° C. In other words, an insoluble beneficial agent complex is a complex which may be dispersed in a vehicle and which is not significantly dissolved in the vehicle. An insoluble beneficial agent complex may include, e.g., a charge-neutralized complex. The term “charge-neutralized complex” is used herein to refer to a complex formed as a result of a non-covalent charge-based interaction between a beneficial agent and an associated molecule, metal, counter ion, etc., and having no net charge or substantially no net charge. Included within this definition are charge neutralized beneficial agents including salts of the beneficial agents.

This complexation contributes to the beneficial release characteristics of the disclosed compositions as discussed herein, e.g., by contributing to the chemical and physical stability of the beneficial agent in the composition, e.g., by reducing degradation of the beneficial agent or providing a complex, which exhibits reduced settling due to gravitational force. In some embodiments, the insoluble beneficial agent complex is formed by including a precipitating and/or stabilizing agent which when combined with the beneficial agent induces formation of an insoluble complex. The insoluble beneficial agent complex may result, for example, from an electrostatic interaction which takes place between the beneficial agent and one or more precipitating and/or stabilizing agents. In some embodiments, the insoluble beneficial agent complex is charge neutralized. Complexation may also reduce a level of chemical conjugation which may occur between the beneficial agent and other components of the formulation, e.g., polymer, in the absence of the complexation.

The insoluble beneficial agent complex according to the present disclosure may be characterized as follows: when 10 mg of the insoluble beneficial agent complex is dispersed and left to stand in 1 mL of a test solution of phosphate buffered saline at pH 7.4 at 37° C. for 24 hours, the amount of beneficial agent dissolved in the test solution is less than 60% of the beneficial agent in the 10 mg of insoluble beneficial agent complex, e.g., less than 50% of the beneficial agent in the 10 mg of insoluble beneficial agent complex, less than 40% of the beneficial agent in the 10 mg of insoluble beneficial agent complex, less than 30% of the beneficial agent in the 10 mg of insoluble beneficial agent complex, or less than 20% of the beneficial agent in the 10 mg of insoluble beneficial agent complex. For example, the amount of beneficial agent dissolved in the test solution may be less than 60% of the beneficial agent in the 10 mg of insoluble beneficial agent complex to about 20% of the beneficial agent in the 10 mg of insoluble beneficial agent complex, e.g., less than 50% of the beneficial agent in the 10 mg of insoluble beneficial agent complex to about 20% of the beneficial agent in the 10 mg of insoluble beneficial agent complex, less than 40% of the beneficial agent in the 10 mg of insoluble beneficial agent complex to about 20% of the beneficial agent in the 10 mg of insoluble beneficial agent complex, or less than 30% of the beneficial agent in the 10 mg of insoluble beneficial agent complex to about 20% of the beneficial agent in the 10 mg of insoluble beneficial agent complex.

In some embodiments, the precipitating or stabilizing agent is a charged species, e.g. a charged molecule, a metal ion or a salt form of a metal ion. Persons having ordinary skill in the art will understand that the salt forms of metal ions are not themselves charged species, but rather provide the source, upon dissociation, of the charged species. For example, in some embodiments, the precipitating agent and/or stabilizing agent is protamine, or a divalent metal ion such as Ni²⁺, Cu²⁺, Zn²⁺, Mg²⁺ and/or Ca²⁺. The divalent metal may be present in the composition as e.g., zinc acetate, zinc carbonate, zinc chloride, zinc sulfate, magnesium acetate, magnesium carbonate, magnesium chloride, magnesium hydroxide, magnesium oxide, magnesium sulfate, calcium acetate, calcium carbonate, calcium chloride, calcium sulfate and the like. That is, the divalent metal salt may be included during preparation of the composition such that a divalent metal salt of the beneficial agent is formed. These precipitating agents and/or stabilizing agents find particular use when the selected beneficial agent is a negatively charged protein or peptide.

It should be noted that the net charge of the beneficial agent may also be adjusted, for example by adjusting the pH. Accordingly, a suitably charged precipitating agent and/or stabilizing agent may be selected based on the net charge of the protein or peptide which may be adjusted. For example, where the beneficial agent has a net positive charge, e.g., as a result of pH adjustment, a negatively charged molecule such as carboxymethylcellulose (CMC) may be utilized as the precipitating agent and/or stabilizing agent.

Thus, some embodiments involve a method of making a complex involving contacting at least one of a protein and peptide with a cationic complexing agent at a pH greater than or equal to 8, e.g., greater than 8.5 or greater than 9, such as 8 to 10, or 8 to 9, to form a complex. Examples of the cationic complexing agent include, but are not limited to, protamine, poly-lysine, poly-arginine, polymyxin, and combinations thereof.

Other embodiments involve a method of making a complex involving contacting at least one of a protein and peptide with an anionic complexing agent at a pH less than or equal to 3, e.g., less than 2.5 or less than 2, such as 1 to 3 or 2 to 3, to form a complex. Examples of the anionic complexing agent include, but are not limited to, carboxy-methyl-cellulose, poly-adenosine, poly-thymine, and combinations thereof.

In some embodiments, following complexing at a specified pH as discussed above, e.g., at a pH greater than or equal to 8 or less than or equal to 3, it may be beneficial to remove supernatant from the mixture formed by contacting the beneficial agent with the complexing agent so at to remove non-complexed, e.g., non-charge-neutralized, beneficial agent, prior to use of the beneficial agent complex in the compositions disclosed herein.

In some embodiments, a cationic agent is complexed with the beneficial agent to form the insoluble beneficial agent complex. Suitable cationic agents may include, but are not limited to, protamine, poly-lysine, poly-arginine, polymyxin, Ca²⁺ and Mg²⁺. Anionic agents may also be utilized as appropriate to form the insoluble beneficial agent complex. Suitable anionic agents may include, but are not limited to, CMC as mentioned above as well as poly-adenosine and poly-thymine. Where the anionic agent is poly-adenosine, the poly-adenosine may be, for example, a 10mer to a 150mer. Where the anionic agent is poly-thymine, the poly-thymine may be, for example, a 10mer to a 1500mer.

Two or more precipitating agents and/or stabilizing agents may be utilized in combination to facilitate formation of the insoluble beneficial agent complexes described herein, e.g., for improved chemical or physical stability of the beneficial agent in the complex and/or improved drug release kinetics, e.g., reduced burst effect and/or a sustained delivery profile. For example, the combination of protamine and a divalent metal or salt thereof with a protein beneficial agent may form an insoluble complex which when dispersed in the vehicle of the disclosed compositions provides a composition having a desired beneficial agent release profile in vivo. In addition, such combinations of precipitating and/or stabilizing agents may improve the chemical and physical stability of the beneficial agent complex and render the complex more resistant to sterilization conditions, e.g., radiation sterilization, including electron beam sterilization and gamma radiation sterilization.

Accordingly, in some embodiments the insoluble beneficial agent complex includes beneficial agent in combination with both protamine and a divalent metal or salt thereof (e.g. Zn²⁺ or Zinc acetate). The molar ratio of beneficial agent:divalent metal or salt:protamine (e.g., beneficial agent:zinc:protamine) may be in the range of 1:0.5 to 2.0:0.3 to 0.5.

Protamine may be used alone or in combination with one of the precipitating agents and/or stabilizing agents described above to form an insoluble beneficial agent complex according to the present disclosure.

The insoluble beneficial agent complexes are present in the composition in the form of insoluble particles. The size of these particles may differ depending on the methods used to prepare the beneficial agent complex. Typically, the particles are small enough to pass through a small needle, such as a 25 gauge needle. In some embodiments the insoluble beneficial agent complex is dispersed in the vehicle in the form of particles having an average size ranging from about 1 μm to about 400 μm in diameter or in largest dimension, e.g., from about 1 μm to about 300 μm, from about 1 μm to about 200 μm, from about 1 μm to about 100 μm, from about 1 μm to about 90 μm, from about 1 μm to about 80 μm, from about 1 μm to about 70 μm, from about 1 μm to about 60 μm, from about 1 μm to about 50 μm, from about 1 μm to about 40 μm, from about 1 μm to about 30 μm, from about 1 μm to about 20 μm, or from about 1 μm to about 10 μm in diameter or in largest dimension. In some embodiments, the insoluble beneficial agent complex is dispersed in the vehicle in the form of particles having an average size ranging from about 10 μm to about 100 μm in diameter or in largest dimension. Particles sizes in this range in combination with density matching, e.g., wherein the density of the particles is the same or similar to the density of the vehicle, contribute to the improved syringeability and injectability of the compositions disclosed herein.

In some embodiments, the density of the insoluble particles is approximately the same as the density of the vehicle in which the particles are dispersed. This provides for increased physical stability of the particles in the vehicle and improved dispersion of the particles in the vehicle particularly during storage of the compositions, e.g., at low temperatures such as 2-8° C. For example, in some embodiments, both the particles and the vehicle have a density of between about 0.9 and 1.2 g/cm³. In some embodiments, the average density of the particles does not differ from that of the vehicle by more than 0.25 g/cm³, e.g., by more than 0.20 g/cm³, by more than 0.15 g/cm³, or by more than 0.05 g/cm³. In some cases, the apparent density of the vehicle is within 10%, e.g., within 8%, within 5%, or within 3%, of the apparent density of the particles.

### Antioxidant as an Additive to Provide Radiation Stability

In some embodiments, e.g., where the composition is to be administered to a human or non-human animal, it may be desirable to include an additive such as antioxidant in order to provide a radiation-stable composition. The addition of antioxidant is particularly important where the beneficial agent is a relatively large molecular weight molecule, e.g., a molecule having a molecular weight greater than 5 kD, e.g., greater than 10 kD, greater than 20 kD, greater than 30 kD, greater than 40 kD, greater than 50 kD, greater than 60 kD, greater than 70 kD, greater than 80 kD, greater than 90 kD or greater than 100 kD. In some embodiments, an antioxidant is added where the beneficial agent is a relatively large molecular weight molecule, e.g., a molecule having a molecular weight from about 5 kD to about 1000 kD, from about 10 kD to about 150 kD, from about 20 kD to about 90 kD, from about 30 kD to about 80 kD, from about 40 kD to about 70 kD, or from about 50 kD to about 60 kD. The addition of antioxidant to an insoluble component, e.g., an insoluble beneficial agent complex, may be useful for example, where the beneficial agent is a protein or a peptide having a molecular weight in the above range. Antioxidant may be added, e.g., to an insoluble component such as an insoluble beneficial agent complex prior to lyophilization or spray-drying to form a powder which can be sterilized, e.g., using a suitable dose of ionizing radiation, either before or after combining the powder with a vehicle as described herein.

Antioxidant may be added at a concentration of from about 0.1 wt % to about 45 wt % of the beneficial agent, e.g., from about 1 wt % to about 35 wt %, from about 5 wt % to about 30 wt %, from about 10 wt % to about 25 wt %, or from about 15 wt % to about 20 wt %. In some embodiments, antioxidant may be added at a concentration of from about 0.1 wt % to about 35 wt % of the beneficial agent, e.g., from about 1 wt % to about 30 wt %, from about 5 wt % to about 25 wt %, from about 10 wt % to about 20 wt %, or at about 15 wt %.

Examples of antioxidants include, but are not limited to, thioethers, histidine, cysteine, tryptophan, tyrosine, ascorbic acid, ascorbic acid palmitate, tocopherols (e.g., vitamin E), butylated hydroxyanisole (BHA), butylated hydroxytoluene (BHT) and mixtures thereof. Thioether antioxidants include, but are not limited to, those disclosed in U.S. Pat. No. 6,989,138, which is incorporated herein by reference in its entirety. While the particular choice of thioether is not limited, one example is methionine. Thioethers include, but are not limited to, methionine. The preferred antioxidant is methionine.

Additional Components

A variety of additional components may be added to the disclosed compositions provided they do not substantially disrupt the beneficial characteristics of the compositions as discussed herein, e.g., viscosity, etc. Suitable components may include, but are not limited to, one or more pharmaceutically acceptable excipients, e.g., stabilizers, dyes, fillers, preservatives, buffering agents, antioxidants, wetting agents, bulking agents, surfactants, anti-foaming agents and the like. Additional components may include, e.g., sucrose, polysorbate, methionine, BHT, tocopherol (e.g., vitamin E), mannitol, trehalose, lactose, Ethylenediaminetetraacetic acid (EDTA) etc. In some embodiments, one or more of the additional components is provided at the same concentration as the active agent. For example, methionine may be included in a composition of the present disclosure as an antioxidant, and in some embodiments sucrose is included as a stabilizer. As discussed above, methionine may also be combined with an insoluble component such as an insoluble beneficial agent complex as described herein to form a radiation stable powder or a radiation stable composition as described herein.

In some embodiments, a high-viscosity carrier such as sucrose acetate isobutyrate (SAIB) may be included in a composition of the present disclosure. For example, SAIB may be included in an amount ranging from about 5% to about 20%, such as about 5% to about 10%, by weight of the vehicle.

In some embodiments, the vehicle comprises about 5% to 10% SAIB, about 70% to about 75% of the hydrophobic solvent, and about 15% to 25% of the biodegradable polymer, wherein each % is % by weight of the vehicle. In one or more embodiments, the vehicle comprises about 5 to about 10% SAIB, about 65% to about 70% benzyl benzoate, about 3% to about 7% ethanol, and about 15% to about 25% poly(lactic-co-glycolic acid) (PLGA), wherein each % is % by weight of the vehicle. In some embodiments, the vehicle comprises about 15% to about 25% SAIB, about 55% to about 65% benzyl benzoate, about 5% to about 15% benzyl alcohol, and about 5% to about 15% polylactic acid (PLA), wherein each % is % by weight of the vehicle. In one or more embodiments, the vehicle comprises about 65% to about 75% benzyl benzoate, about 5% to about 15% benzyl alcohol, and about 15% to about 25% polylactic acid (PLA), wherein each % is % by weight of the vehicle.

In one or more embodiments, inclusion of SAIB at 8% by weight of the vehicle, allows for inclusion of the hydrophobic solvent at 72%, by weight of the vehicle and inclusion of the biocompatible, biodegradable polymer at 20% by weight of the vehicle. In some embodiments, the amount of SAIB in the composition may be adjusted provided that the weight % of the hydrophobic solvent is maintained between about 60 and about 95% by weight of the vehicle and the weight % of the biocompatible, biodegradable polymer is maintained between about 5 and about 40% by weight of the vehicle.

For instance, the amount of SAIB may be adjusted from 0 to 35% by weight of the vehicle, e.g., in 1% intervals, provided that the percentages of the hydrophobic solvent and the biocompatible, biodegradable polymer are adjusted accordingly, preferably provided that the zero shear viscosity of the resulting composition does not exceed 1,200 cP at 25° C. Without reciting each combination of the above three components that fall within the specified ranges, it is to be understood that all such combinations are within the scope of the present disclosure and further that this is intended to provide antecedent basis for specific recitations of any combination of the above three components that meet the above range and viscosity recitations.

Methods of Preparation

In general, the present compositions may be made by any of the various methods and techniques known and available to those skilled in the art.

The compositions of the present disclosure may be prepared generally by combining a biodegradable polymer as described herein and a hydrophobic solvent as described herein to form a vehicle of the composition. The biodegradable polymer is typically provided in an amount of from about 5% to about 40% by weight of the vehicle, and the hydrophobic solvent is typically provided in an amount of from about 95% to about 60% by weight of the vehicle. The insoluble component comprising beneficial agent, e.g., an insoluble beneficial agent complex, is dispersed in the vehicle. Such dispersion may occur following one or more milling or sieving steps to obtain particles of a desired size. One or more homogenization steps may be utilized following dispersion of the insoluble beneficial agent or insoluble beneficial agent complex in the vehicle. It should be noted that within the above ranges the % by weight of the biodegradable polymer and the hydrophobic solvent may be adjusted while maintaining a desired viscosity range, e.g., a zero shear viscosity less than 1,200 centipoise (cP), e.g., less than 1000 cP, less than 500 cP or less than 100 cP at 25° C., such as a zero shear viscosity less than 1,200 centipoise to about 100 cP, e.g., less than 1000 cP to about 100 cP, or less than 500 cP to about 100 cP at 25° C. In addition, one or more additional components may be included in the vehicle as described previously herein.

Insoluble beneficial agent complex particles may be prepared, for example, by dissolving the beneficial agent in a suitable buffer and subsequently adding a suitable amount of a stabilizing/precipitating agent (e.g., a precipitating agent and/or stabilizing agent as described herein, e.g., protamine, zinc, etc.) until a precipitate is formed at a temperature greater than the freezing point but less than the boiling point of the buffer. The suitable buffer with dispersed precipitate is then subjected to a suitable drying process, e.g., spray drying or lyophilization, to provide a powder comprising insoluble beneficial agent complex. Alternatively, the precipitate can be recovered by centrifugation and removal of the resulting supernatant. It can then be re-suspended in aqueous medium for spray drying or lyophilized directly. The particles may also be formed through liquid nitrogen quenching or supercritical fluid processing. One or more size reduction and sieving steps may be utilized to adjust the particle size of the beneficial agent complex. The complexed powder is mixed with a suitable amount of the prepared vehicle to disperse the beneficial agent complex particles in the vehicle. In some embodiments, where the beneficial agent is a low molecular weight compound, the beneficial agent complex may include only the salt form of the beneficial agent, provided that the salt form of the beneficial agent is at least substantially insoluble in the vehicle. The formulation may be sterilized prior to use using any suitable method known in the art, e.g., sterilization with an ionizing radiation such as gamma irradiation, e-beam radiation or x-ray radiation, at a dose of about 10 kGy to about 25 kGy. Alternatively, the beneficial agent complex and the vehicle may be sterilized separately and then combined prior to use.

Suitable forms of ionizing radiation include, e.g., gamma radiation, e-beam radiation and x-ray radiation. One of ordinary skill in the art will be able to determine an appropriate sterilizing dose of radiation based on a variety of factors including, e.g., the type of radiation, the shape, size, and/or composition of the material to be sterilized, the desired level of sterility and the amount of contamination present prior to sterilization. The irradiation may be conducted with the beneficial agent complex and/or the vehicle maintained at from about 0° C. to about 30° C., e.g., from about 0° C. to about 5° C., from about 5° C. to about 10° C., from about 10° C. to about 15° C., from about 15° C. to about 20° C., from about 20° C. to about 25° C., or from about 25° C. to about 30° C.

In some embodiments, a suitable dose of sterilizing radiation is a dose of about 10 kGy to about 25 kGy, e.g., about 15 kGy to about 20 kGy.

In some embodiments, when stored at 2° C., 8° C., or 25° C., the composition maintains a purity of at least 90% or greater (e.g., at least 95% or greater) for a period of at least 24 hours following exposure to gamma irradiation at a dose of about 10 kGy to about 25 kGy, e.g., about 15 kGy to about 20 kGy. For example, the period may be from about 24 hours to about 48 hours, from about 48 hours to about 72 hours, from about 72 hours to about 96 hours, from about 96 hours to about 120 hours, from about 120 hours to about 144 hours, from about 144 hours to about 168 hours, from about 168 hours to about 192 hours, from about 192 hours to about 216 hours, from about 216 hours to about 240, from about 240 hours to about 264 hours, from about 264 hours to about 288 hours, from about 288 hours to about 312 hours, from about 312 hours to about 336 hours, from 336 hours to about 360 hours, from about 360 hours to about 384 hours, from about 384 hours to about 408 hours, from about 408 hours to about 432 hours, from about 432 hours to about 456 hours, from about 456 hours to about 480 hours, from about 480 hours to about 504 hours, from about 504 hours to about 528 hours, from about 528 hours to about 552 hours, from about 552 hours to about 556 hours, from about 556 hours to about 600 hours, from about 600 hours to about 624 hours, from about 624 hours to about 648 hours, from about 648 hours to about 672 hours, from about 672 hours to about 696 hours, or from about 696 hours to about 720 hours. The period may also be 3 months, 6 months, 1 year, or 2 years. In some embodiments, a purity of at least 90% or greater (e.g., 95% or greater) is maintained for a period of at least one month, e.g., following exposure to gamma irradiation at a dose of about 10 kGy to about 25 kGy, e.g., about 15 kGy to about 20 kGy. For example, the period may be from about one month to about two months, from about two months to about three months, from about three months to about four months, from about four months to about five months, from about five months to about six months, from about six months to about one year, or from about one year to about two years.

In some embodiments, where the composition maintains a purity of at least 90% or greater (e.g., at least 95% or greater) for a time period indicated above following exposure to gamma irradiation at a dose of about 10 kGy to about 25 kGy, e.g., about 15 kGy to about 20 kGy, the composition is maintained at a temperature of from about 0° C. to about 30° C. for the time period, e.g., from about 0° C. to about 5° C., from about 5° C. to about 10° C., from about 10° C. to about 15° C., from about 15° C. to about 20° C., from about 20° C. to about 25° C., or from about 25° C. to about 30° C. In some embodiments, where the composition maintains a purity of at least 90% or greater (e.g., at least 95% or greater) for a time period indicated above following exposure to gamma irradiation at a dose of about 10 kGy to about 25 kGy, e.g., about 15 kGy to about 20 kGy, the composition is maintained at a temperature of about 25° C. for the time period.

Purity may be determined, for example, based on Reverse Phase High Pressure Liquid Chromatographic (RPLC) analysis of the composition. For example, RPLC spectra for the active agent in the irradiated composition can be compared with RPLC spectra for a USP standard of the active agent. Peak retention times for the active agent in the irradiated composition can be matched to the USP standard for the active agent, and impurity peaks can be subtracted to obtain % purity levels.

Biodegradable Formulations

As discussed previously herein, in some embodiments, the biodegradable compositions of the present disclosure include A) a single phase vehicle including i) a biodegradable polymer present in an amount of from about 5% to about 40% (e.g., from about 6% to about 29%, from about 7% to about 28%, from about 8% to about 27%, from about 9% to about 26%, from about 10% to about 25%, from about 11% to about 24%, from about 12% to about 23%, from about 13% to about 22%, from about 14% to about 21%, from about 15% to about 20%, from about 16% to about 19%, or from about 17% to about 18%) by weight of the vehicle, and ii) a hydrophobic solvent present in an amount of from about 95% to about 60% (e.g., from about 94% to about 61%, from about 93% to about 62%, from about 92% to about 63%, from about 91% to about 64%, from about 90% to about 65%, from about 89% to about 66%, from about 88% to about 67%, from about 87% to about 68%, from about 86% to about 69%, from about 85% to about 70%, from about 84% to about 71%, from about 83% to about 72%, from about 82% to about 73%, from about 81% to about 74%, from about 80% to about 75%, from about 79% to about 76%, or from about 78% to about 77%) by weight of the vehicle; and B) an insoluble component comprising beneficial agent, e.g., an insoluble beneficial agent complex, dispersed in the vehicle, wherein the biodegradable composition has a zero shear viscosity less than 1,200 centipoise (cP) (e.g., less than 1100 cP, less than 1000 cP, less than 900 cP, less than 800 cP, less than 700 cP, less than 600 cP, less than 500 cP, less than 400 cP, less than 300 cP, less than 200 cP, or less than 100 cP) at 25° C., is injectable through a small gauge needle and is not an emulsion or gel. For example, the biodegradable composition may have a zero shear viscosity of from less than 1,200 cP to about 1100 cP, less than 1100 cP to about 1000 cP, less than 1000 cP to about 900 cP, less than 900 cP to about 800 cP, less than 800 cP to about 700 cP, less than 700 cP to about 600 cP, less than 600 cP to about 500 cP, less than 500 cP to about 400 cP, less than 400 cP to about 300 cP, less than 300 cP to about 200 cP, or less than 200 cP to about 100 cP at 25° C., wherein the biodegradable composition is injectable through a small gauge needle and is not an emulsion or gel.

In some embodiments, a biodegradable composition of the present disclosure has a zero shear viscosity less than 1,200 cP (e.g., less than 1100 cP, less than 1000 cP, less than 900 cP, less than 800 cP, less than 700 cP, less than 600 cP, less than 500 cP, less than 400 cP, less than 300 cP, less than 200 cP, or less than 100 cP) at 25° C.

It should be noted that the amount of the biodegradable polymer and the amount of the hydrophobic solvent may be varied, for example, to achieve a desired viscosity, e.g., in 1% by weight increments, provided that they are typically maintained within about 5% to about 40% by weight of the vehicle and about 95% to about 60% by weight of the vehicle, respectively.

Accordingly, without reciting every possible combination falling within the above ranges, this is intended to provide antecedent basis for such combinations.

In some embodiments, the zero shear viscosity of the biodegradable composition is from about 1000 cP to about 100 cP, e.g., about 900 cP to about 100 cP, about 800 cP to about 100 cP, about 700 cP to about 100 cP, about 600 cP to about 100 cP, about 500 cP to about 100 cP, about 400 cP to about 100 cP, about 300 cP to about 100 cP, or about 200 cP to about 100 cP at 25° C.

In some embodiments, in addition to a relatively low viscosity at 25° C., the disclosed biodegradable compositions also exhibit relatively low viscosity at 37° C., e.g., a zero shear viscosity less than 500 cP, less than 400 cP, less than 300 cP, less than 200 cP, or less than 100 cP. In some embodiments, the zero shear viscosity of the biodegradable composition is from about 500 cP to about 100 cP, from about 400 cP to about 200 cP, or about 300 cP at 37° C. The viscosity of these formulations declines with increasing temperature; frequently in exponential fashion.

The disclosed biodegradable compositions also typically exhibit relatively low viscosity (e.g., a zero shear viscosity less than 500 cP, less than 400 cP, less than 300 cP, less than 200 cP, or less than 100 cP) at 37° C. after being exposed to phosphate-buffered saline in vitro, and maintain this low viscosity over time, e.g., for at least 5 hrs, at least 24 hrs, at least 48 hrs, at least 72 hrs, or at least 168 hrs, of exposure to phosphate-buffered saline. For example, the disclosed biodegradable compositions typically exhibit relatively low viscosity (e.g., a zero shear viscosity of less than 500 cP to about 100 cP, less than 400 cP to about 100 cP, less than 300 cP to about 100 cP, or less than 200 cP to about 100 cP) at 37° C. after being exposed to phosphate-buffered saline in vitro, and maintain this low viscosity over time, e.g., from 5 hrs to about 24 hrs, from about 24 hrs to about 48 hrs, from about 48 hrs to about 72 hrs, or from about 72 hrs to about 168 hrs of exposure to phosphate-buffered saline.

Surprisingly, the disclosed biodegradable depot compositions typically demonstrate good syringeability and injectability while providing for sustained release of the beneficial agent in-vivo with minimal burst. Syringeability and injectability may be characterized by the time it takes to inject a known volume of the biodegradable depot composition through a syringe of known size fitted with a relatively small gauge needle, e.g., a 1-5 mL syringe fitted with a needle having a gauge of about 21 to about 27. In some embodiments, the biodegradable depot compositions of the present disclosure may be characterized as having good syringeability and injectability based on their ability to be injected through a 1 ml syringe fitted with an approximately 0.5 in needle having a gauge of about 21 to about 27, wherein a 0.5 ml volume of the biodegradable depot can be injected in less than 25 sec (e.g., less than 20 sec., less than 15 sec, less than 10 sec, or less than 5 sec) at 25° C. with the application of a 5 to 10 lb force. In some embodiments, under the above conditions, the biodegradable depot can be injected in a range of from about 25 sec to about 1.5 sec, e.g., from about 20 sec to about 1.5 sec, from about 15 sec to about 1.5 sec, from about 10 sec to about 1.5 sec, or from about 5 sec to about 1.5 sec.

In addition to good injectability and syringeability as described herein, in some embodiments, the biodegradable compositions of the present disclosure demonstrate minimal burst and sustained delivery of beneficial agent over time. “Minimal burst” may be characterized in terms of Cₘₐₓ/Cₘᵢₙ, wherein the acceptable Cₘₐₓ/Cₘᵢₙ upper limit may vary depending on the beneficial agent to be delivered. In some embodiments, the weight % of beneficial agent released as burst over the first 24 hours is less than 30% of the total amount released over one week, e.g., less than 20% or less than 10%, of the total amount released over one week. For example, the weight % of beneficial agent released as burst over the first 24 hours may be less than 30% to about 20% or from about 20% to about 10%, of the total amount released over one week. In some embodiments, the weight % of beneficial agent released as burst over the first 24 hours is less than 10% of the total amount released over one month, e.g., less than 8% or less than 5%, of the total amount released over one month. For example, the weight % of beneficial agent released as burst over the first 24 hours may be less than 10% to about 8% or from about 8% to about 5%, of the total amount released over one month. As used herein, “sustained delivery” refers to durations which are at least several fold, e.g., at least 5 fold to at least 10 fold, longer than the duration obtained from a single dose of an immediate-release (IR) formulation of the same beneficial agent (determined by Adsorption, Distribution, Metabolism, and Excretion (ADME) characteristics of the beneficial agent itself).

As mentioned above, the disclosed biodegradable compositions provide for sustained release of the beneficial agent in-vivo with minimal burst effect in addition to possessing good injectability, syringeability and chemical stability as discussed above. This is an unexpected and surprising result as currently available formulations generally provide either controlled release or injectability/syringeability but not both. For example, commercially available depot formulations may rely on the formation of an extremely viscous polymer matrix to provide controlled release of a beneficial agent. However, such formulations have poor injectability/syringeability due to the viscous nature of the depot. Alternatively, other commercially available formulations utilize vehicles which may have good injectability/syringeability due to a high-solvent content but poor control over release of the beneficial agent. Moreover, one would expect a low viscosity liquid composition such as those disclosed herein to have poor release kinetics in the form of a substantial burst effect and an exponentially declining delivery profile. Contrary to this expectation, the present compositions demonstrate low burst effect and good control over release of the beneficial agent over a period of one day to one month or longer.

Without intending to be bound by any particular theory, it is believed that the beneficial release characteristics of the compositions of the present disclosure are due at least in part to the formation of a fluid, non-structured (without any appreciable mechanical integrity), “rate-controlling cloud” or “rate-controlling film” at the surface of the composition in vivo. The rate-controlling cloud or film can be characterized as occurring at the surface of the composition in the aqueous environment. The desirable controlled delivery characteristic of the disclosed compositions may result from the rate-controlling contributions of both the insoluble component comprising beneficial agent, e.g., an insoluble beneficial agent complex, dispersed in the liquid core of the composition and the polymer cloud or film on the surface of the composition. In addition, in some embodiments, a synergistic effect with respect to release rate control, e.g., as demonstrated by MRT, is seen as an apparent result of interaction between the beneficial agent complex and the rate controlling cloud or film. While the rate controlling cloud or film lacks appreciable mechanical integrity, it has a measurable thickness less than 10 μm.

In some embodiments, the compositions of the present disclosure lack gel forming or gelling characteristics. For example, many prior art vehicle compositions exhibit gel formation when aged at 37° C. which can be characterized by an increase in the storage modulus relative to the loss modulus. In contrast, the compositions of the present disclosure can be characterized by a relatively large G″/G′ ratio, e.g., a G″/G′ ratio of greater than or equal to 10, such as greater than or equal to 15 or greater than or equal to 20, following aging at 37° C. for a period of 14 days, wherein G″ is the loss modulus and G′ is the storage modulus.

In certain embodiments, the compositions are Newtonian. For instance, in some cases, the viscosity of the composition at 25° C. varies less than 7%, less than 6%, less than 5%, less than 4%, or less than 3%, when measured at a shear rate ranging from 7 sec⁻¹ to 500 sec⁻¹. For example, the viscosity of the composition at 25° C. may vary from less than 6% to about 3%, from about 5% to about 3%, or from about 4% to about 3%, when measured at a shear rate ranging from 7 sec⁻¹ to 500 sec⁻¹.

Without intending to be bound by any particular theory, a composition comprising a charge-neutralized complex of a beneficial agent containing acid groups such as a peptide or protein may be characterized as follows. During the event of charge neutralization, either peptide or protein or any acid terminated molecule can become negatively charged at basic pH (pH>8) in the presence of buffer. The charged beneficial molecule in aqueous solution will be neutralized with solution of positively charged counter-ion such as protamine or Zn²⁺ ion at an optimal molar ratio. This molar concentration of either protamine or zinc ion is obtained by titration of protamine or zinc ion against the fixed concentration of negatively charged peptide or protein. The molar concentration of either protamine or zinc ion will also depend on the net charge on the protein or peptide and its molar concentration. The aqueous solubility of charge-neutralized complex (peptide or protein plus counter-ion) is dramatically reduced and it will precipitate out of solution. Any charged species of protein or peptide and counter-ion remain in the solution. The dried powder of insoluble beneficial agent—counter-ion complex can be uniformly dispersed in a polymer solution (vehicle) either by hand or mechanical mixing (e.g. homogenization). The resultant formulation controls the release of the beneficial agent via solubility, dissolution rate, and diffusivity. Electrostatic, hydrogen bonding and hydrophobic interactions may also occur between the dispersed particles of charge-neutralized beneficial agent and polymer, and these may also modulate the release kinetics as manifested by the surprising contribution by the polymer-complex interaction to MRT of the beneficial agent in vivo.

In some embodiments, the disclosed compositions are suspensions that remain substantially homogenous for about 3 months, even more preferably for about 6 months, and yet even more preferably, for about 1 year. In one or more embodiments, the insoluble beneficial agent complex remains physically and chemically stable in the suspension vehicle for about 3 months, even more preferably for about 6 months, and yet even more preferably, for about 1 year.

Administration of Biodegradable Formulations

As discussed previously herein, the disclosed biodegradable formulations possess low viscosity along with good injectability and syringeability making them well suited for delivery via a syringe (e.g., a 1-5 mL syringe) with a needle, e.g., 18 gauge to 27 gauge, such as a narrow gauge needle, e.g., 21 to 27 gauge. In addition, the injectable depot formulations may also be delivered via one or more needleless injectors known in the art.

Suitable routes of administration include, but are not limited to, subcutaneous injection and intramuscular injection. Suitable routes of administration also include, for example, intra-articular and intra-ocular, e.g., intra-vitreal, administration for local delivery.

The formulations disclosed herein may also find use in oral formulations, e.g., formulations delivered in a gel-cap (soft or hard) or as a mouthwash.

The formulations disclosed herein may also find use as coatings for medical devices, e.g., implantable medical devices. Such coatings may be applied, e.g., by dip-coating the medical device prior to implantation.

The formulations of the present disclosure may be formulated such that a desired pharmacological effect is achieved via administration on a periodic basis. For example, the formulations may be formulated for administration on a daily, weekly or monthly basis.

The actual dose of the beneficial agent or insoluble beneficial agent complex to be administered will vary depending on the beneficial agent, the condition being treated, as well as the age, weight, and general condition of the subject as well as the severity of the condition being treated, and the judgment of the health care professional. Therapeutically effective amounts are known to those skilled in the art and/or are described in the pertinent reference texts and literature.

For example, in the case of proteins and peptides beneficial agents, the beneficial agent will typically be delivered such that plasma levels of the beneficial agent are within a range of about 5 picomoles/liter to about 200 picomoles/liter. On a weight basis, a therapeutically effective dosage amount of protein or peptide will typically range from about 0.01 mg per day to about 1000 mg per day for an adult. For example, peptide or protein dosages may range from about 0.1 mg per day to about 100 mg per day, or from about 1.0 mg per day to about 10 mg/day.

In some embodiments, a suitable low molecular weight compound may be characterized as one which can provide the desired therapeutic effect with a dose of less than or equal to about 30 mg/day as delivered from a depot administered once a week, or a dose of less than or equal to about 10 mg/day as delivered from a depot administered once a month. For example, a suitable low molecular weight compound may be one which can provide the desired therapeutic effect with a dose of less than about 30 mg/day, e.g., less than about 25 mg/day, less than about 20 mg/day, less than about 15 mg/day, less than about 10 mg/day, less than about 5 mg/day or less than about 1 mg/day as delivered from a depot administered once a week. In some embodiments, a suitable low molecular weight compound is one which can provide the desired therapeutic effect with a dose of from about 30 mg/day to about 1 mg/day, e.g., from about 25 mg/day to about 5 mg/day, or from about 20 mg/day to about 10 mg/day as delivered from a depot administered once a week.

Similarly, a suitable low molecular weight compound may be one which can provide the desired therapeutic effect with a dose of less than about 10 mg/day, less than about 9 mg/day, less than about 8 mg/day, less than about 7 mg/day, less than about 6 mg/day, less than about 5 mg/day, less than about 4 mg/day, less than about 3 mg/day, less than about 2 mg/day or less than about 1 mg/day as delivered from a depot administered once a month. In some embodiments, a suitable low molecular weight compound may be one which can provide the desired therapeutic effect with a dose of from about 10 mg/day to about 1 mg/day, e.g., from about 9 mg/day to about 2 mg/day, from about 8 mg/day to about 3 mg/day, from about 7 mg/day to about 4 mg/day, or from about 6 mg/day to about 5 mg/day as delivered from a depot administered once a month.

In some embodiments, e.g., where the formulation may have been in storage for a period of time prior to injection, the formulation may be mixed, e.g., via shaking, prior to administration to ensure that the insoluble component comprising beneficial agent, e.g., an insoluble beneficial agent complex, is sufficiently dispersed in the vehicle carrier.

In some embodiments the depot formulations disclosed herein (or components thereof) are sterilized prior to use, e.g., via the application of a sterilizing dose of ionizing radiation. For example, in one embodiment one or both of the insoluble beneficial agent complex and a vehicle as disclosed herein are sterilized with ionizing radiation, e.g., gamma radiation, e-beam radiation, or x-ray radiation, prior to combining to form a depot composition as disclosed herein. In an alternative embodiment, the insoluble beneficial agent complex and the vehicle may be combined and the suspension may be subjected to radiation sterilization.

Kits

A variety of kits may be provided which include one or more components of the biodegradable formulations disclosed herein along with instructions for preparing and/or using the same. For example, in one embodiment, a suitable kit may include a vehicle as described herein in a first container and an insoluble component comprising beneficial agent, e.g., an insoluble beneficial agent complex, as described herein in a second container, e.g., in powder form. These components may then be mixed together prior to injection to form a biodegradable formulation according to the present disclosure. In some embodiments, the first container is a syringe which may be coupled to the second container, e.g., a vial with a luer lock, to provide a mechanism for mixing the vehicle and the insoluble component comprising beneficial agent, e.g., an insoluble beneficial agent complex. In other embodiments, both the first and second containers are syringes which may be coupled, e.g., via a luer lock, to provide a mechanism for mixing the vehicle and the insoluble component comprising beneficial agent, e.g., an insoluble beneficial agent complex.

In another embodiment, the biodegradable formulation may be provided pre-mixed in a single container, e.g., a single syringe.

In another embodiment, the biodegradable formulation may be provided un-mixed in a pre-filled, dual-chamber syringe including a first chamber containing the vehicle and a second chamber containing the insoluble component comprising beneficial agent, e.g., an insoluble beneficial agent complex. The syringe may be provided such that a user can initiate contact and subsequent mixing of the vehicle and the insoluble component comprising beneficial agent, e.g., an insoluble beneficial agent complex.

The instructions for use of the kit and/or kit components may be provided as complete written instructions along with the kit, e.g., as an insert card or printed on the kit packaging; or stored on a computer readable memory device provided with the kit. Alternatively, the kit may include instructions which provide a brief instruction to the user and direct the user to an alternate source for more complete use instructions. For example, the kit may include a reference to an internet site where the complete instructions for use may be accessed and/or downloaded.

### EXAMPLES

The following examples are put forth so as to provide those of ordinary skill in the art with a disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight as measured by gel permeation chromatography, temperature is in degrees Celsius, and pressure is at or near atmospheric. Standard abbreviations may be used, e.g., bp, base pair(s); kb, kilobase(s); kd, kiloDalton(s); pL, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1: Stability of Radiation-Sterilized hGH Formulations

### Materials and Methods

Preparation of hGH Powder:

Aliquots of 3 mL each of the bulk hGH solution in buffer from BresaGen were transferred into 5 mL type-Hypak BD glass syringes and lyophilized using the lyophilization cycle provided in Table 1 (optimized for hGH) to fit the steps provided with an FTS lyophilizer, Dura Stop, MP Stoppering Tray Dryer, Stone Ridge, N.Y.

Preparation of hGH: Protamine with or without Methionine Powder:

100 mg of BresaGen hGH powder was placed in a 15 mL wide-mouth glass jar. 5.5 mL of 25 mM NH₄HCO₃ (pH˜7.5) solution was added and the compound was stirred for 30 min at room temperature, 400 rpm until it became clear. Then 1.02 mL of protamine sulfate solution (conc. 10 mg/mL) was slowly added to form a white precipitate. The resulting suspension was stirred for 30 min to complete the complexation reaction. 0.19 mL of 290 mM sucrose solution was then added while stirring at 400 rpm. When the solution was clear, 15.2 μL of 10% polysorbate 20 solution was added. In powders with methonine formulation, 1 wt %-35 wt % of methionine relative to the total protein weight was added to the complexed suspension. Aliquots of 3 mL each of the bulk suspension from the above step were transferred into 5 mL type-Hypak BD glass syringes and lyophilized using the lyophilization cycle provided in Table 1 (optimized for hGH) to fit the steps provided with an FTS lyophilizer, Dura Stop, MP Stoppering Tray Dryer, Stone Ridge, N.Y. Some of the aliquots from the above suspension were spray-dried using Buchi B329 spray dryer using N2 as the carrier gas.

The spray-dry conditions were as follows: Inlet temperature set up: 140° C., Aspirator 100%, Pump: 13%, Nozzle Cleaner: 2 pulses per min.

TABLE 1

| | SHELF | | Chamber | |
|---|---|---|---|---|
| | TEMP | TIME | pressure | |
| STEP | (° C.) | (HOUR) | (mTorr) | Modifications |
| FREEZING | PRECOOL @ −40 | | Not | 1 hr prior loading |
| | | | controlled | Instrument was |
| | | | | pre-cooled |
| | −40 | 2.0 | 3000 | Modified to fit the |
| | | | | freezing steps |
| | | | | available for FTS |
| | | | | Lyophilizer |
| PRIMARY | −25 | 2.0 | 100 | |
| DRYING | −30 | 35.0 | | |
| SECONDARY | 25 | 2.0 | | Actual time was 24.7 |
| DRYING | | | | hours due to the slight |
| | | | | variation of ramping |
| | | | | speed (setting is 2 |
| | | | | digits after decimal |
| | | | | point) |
| | 25 | 10.0 | | |
| | 5 | 10.0 | 200 | Actual hold time was |
| | | | | 8 hours |

Gamma Irradiation of Formulations:

Lyophilized or spray-dried powders of hGH stabilized with protamine were filled in either syringe with stopper or vial targeting 50 mg dose each. In a two-component system, lyophilized or spray-dried powders were filled in syringe with stopper and sealed in an aluminum pouch. Vehicles of SAM/BB/PLA (8/72/20, % w/w) were filled in a vial, stoppered and sealed in an aluminum pouch separately. In a one-component system, lyophilized or spray-dried powders were placed in a vial targeting 50 mg of hGH per dose and 1 mL of SAM/BB/PLA (8/72/20, % w/w) was added and homogenized for 10 min as a final suspension. The vial containing the suspension was stoppered and sealed in an aluminum pouch. One-component and two-component package were sent to Gamma-STAT facility of Sterigenics, Corona, Calif. in a refrigerated package. One-component and two-component systems were exposed to ambient condition (facility room temperature ˜25° C. and exposed to either no-radiation or 2.5-7.5 kGy (targeting 5 kGy) or 7.5-12.5 kGy (targeting 10 kGy) or 12.5-17.5 kGy (targeting 15 kGy) or 22.5-27.5 kGy (targeting 25 kGy) respectively. Samples of hGH powder formulation without complex were also exposed to the different doses of Gamma-radiation as controls.

Stability of the above formulations was determined using RPLC analysis. The results are provided in Tables 2-5 below. Results are shown below for spray-dried formulations but formulations prepared using lyophilization showed similar results. Peak retention times were matched to a USP standard for hGH and impurity peaks were subtracted to obtain % purity levels.

Results

Table 2 shows the Gamma-radiation Stability of the hGH:Protamine complex powder without methionine as an additive

TABLE 2

| Gamma- | Spray-dried | |
|---|---|---|
| Irradiation | (SD) | % Purity |
| No | SD | 98 |
| Yes (9.6 kGy) | SD | 87 |
| Yes (14.5 kGy) | SD | 85 |

The results shown in Table 2 indicate that treatment with Gamma-radiation at doses up to 14.5 kGy results in degradation of the hGH protein even in the presence of protamine as a complexing agent. Stability was analyzed at Time 0 following receipt of the gamma-irradiated formulations.

Table 3 shows the Gamma-radiation Stability of the hGH:Protamine complex powder with methionine (35 wt % to hGH) as an additive.

TABLE 3

% Purity

| Gamma- | Spray-dried | T = | T = |
|---|---|---|---|
| Irradiation | (SD) | 0 hr | 1 month |
| No | SD | 100 | 96 |
| Yes (3.8 kGy) | SD | 100 | ND |
| Yes (9.6 kGy) | SD | 97 | ND |
| Yes (14.5 kGy) | SD | 95 | 95 |
| Yes (23.4 kGy) | SD | 95 | 95 |

ND = not determined

The results shown in Table 3 indicate that when methionine is included at 35 wt % of hGH in the hGH:protamine complex powder prior to spray-drying, hGH stability is retained out to 1 month (when stored at 2-8° C.) following treatment with Gamma-radiation at doses up to 23.4 kGy. Thus, in order to increase the stability (in terms of % purity) of a relatively high molecular weight protein such as hGH when treating with high exposure Gamma-radiation, it is important to include both a complexing agent such as protamine and methionine.

Table 4 shows the Gamma-radiation Stability of hGH:Protamine complex powder in SAM/BB/PLA (8/72/20, % w/w) without Methionine.

TABLE 4

| Gamma- | Spray-dried | |
|---|---|---|
| Irradiation | (SD)/1-component | % Purity |
| No | SD | 94 |
| Yes (9.6 kGy) | SD | 82 |
| Yes (14.5 kGy) | SD | 70 |

The results shown in Table 4 indicate that the stability of hGH in the hGH:Protamine complex powder is reduced when the spray-dried powder is suspended in the SAM/BB/PLA (8/72/20, % w/w) vehicle prior to treatment with Gamma-radiation at doses up to 14.5 kGy. The presence of the protamine complex alone in the formulation is not sufficient to maintain the stability (in terms of % purity) of the hGH in the formulation. Stability was analyzed at Time 0 following receipt of the gamma-irradiated formulations.

Table 5 shows the Gamma-radiation Stability of hGH:Protamine complex powder in SAM/BB/PLA (8/72/20, % w/w) with methionine (35 wt % to hGH) as an additive.

TABLE 5

| | | % Purity | | |
|---|---|---|---|---|
| Gamma- | Spray-dried | T = | T = | T = |
| Irradiation | (SD)/1-component | 0 hr | 1 week | 1 month |
| No | SD | 100 | N/D | 96 |
| Yes (3.8 kGy) | SD | 97 | 97 | N/D |
| Yes (9.6 kGy) | SD | 96 | N/D | N/D |
| Yes (14.5 kGy) | SD | 96 | N/D | 95 |
| Yes (23.4 kGy) | SD | 95 | N/D | 93 |

ND = not determined

The results shown in Table 5 indicate that when methionine is included at 35 wt % of hGH in the hGH:protamine complex powder prior to spray-drying and suspending in the SAM/BB/PLA (8/72/20, % w/w) vehicle, hGH stability is retained out to 1 month (when stored at 2-8° C.) following treatment with Gamma-radiation at doses up to 23.4 kGy. Thus, in order to increase the stability (in terms of % purity) of a relatively high molecular weight protein such as hGH in a formulation including a SAM/BB/PLA (8/72/20, % w/w) vehicle, when treating with high exposure Gamma-radiation, it is important to include both a complexing agent such as protamine and methionine.

### Example 2: Stability of Radiation-Sterilized Nucleoside Formulations

Materials and Methods

Preparation of Nucleoside Powder:

Bulk nucleoside powder (485 mg) was diluted with 10 mL of MilliQ water and stirred with a magnetic stirrer bar on a stirrer plate for 15 min at 2-8° C. 25 mL of 10 mg/mL protamine sulfate was added forming a suspension. The suspension was then stirred for another 15 min. Aliquots of 3 mL each of the bulk solution were transferred into 5 mL, type-Hypak BD glass syringes and lyophilized using the lyophilization cycle shown below in Table 6 (optimized for protamine sulfate) to fit the steps provided with FTS lyophilizer, Dura Stop, MP Stoppering Tray Dryer, Stone Ridge, N.Y. The syringes were seal pouched and stored in a −20° C. freezer until further study. The final composition of the lyophilized powder was about 41.5 mg of nucleoside and about 21.4 mg of protamine sulfate per syringe.

TABLE 6

| | SHELF | | Chamber | |
|---|---|---|---|---|
| | TEMP | TIME | pressure | |
| STEP | (° C.) | (HOUR) | (mT) | Modification |
| FREEZING | PRECOOL @ −40 | | Not | Instrument was |
| | | | controlled | pre-cooled 1 hr prior |
| | | | | loading |
| | −40 | 2.0 | 3000 | Modified to fit the |
| | | | | freezing steps |
| | | | | available for FTS |
| | | | | Lyophilizer |
| PRIMARY | −25 | 2.0 | 100 | |
| DRYING | −30 | 35.0 | | Due to power failure, |
| | | | | actual hold time was |
| | | | | 40 hours. |
| SECONDARY | 25 | 2.0 | | Actual time was 24.7 |
| DRYING | | | | hours due to the slight |
| | | | | variation of ramping |
| | | | | speed (setting is 2 |
| | | | | digits after decimal |
| | | | | point) |
| | 25 | 10.0 | | |
| | 5 | 10.0 | 200 | Actual hold time was |
| | | | | 8 hours |

Preparation of Nucleoside Active Agent Powder in Vehicle and Gamma Irradiation of Same:

0.5 mL of a SAM/BB/EtOH/PLGA (8/67/5/20, % w/w) vehicle in 1 mL syringes was mixed with the lyophilized nucleoside powder (described above) in 3 mL glass syringes. The PLGA was dodecanol initiated, had a weight average molecular weight of 6.5 kD, and had an L:G molar ratio of 65:35. The vehicle was added into 90 mg of the powder and mixed back and forth until it was homogeneous. The formulations were then exposed to a dose of about 25 kGy of Gamma radiation at about 25° C. The syringes were then stored at 2-8° C.

The purity of the nucleoside in the stored syringes was determined with respect to time as follows. At each time point, the complexed nucleoside active agent was extracted from the vehicle as follows. 1 ml of ethylacetate was added to each syringe. Each syringe was then vortexed and centrifuged to remove the vehicle containing supernatant. The complexed nucleoside active agent pellet was dried and then dissolved in 2% H3PO4 in water to separate the complexing agent from the active agent prior to running RPLC. The RPLC conditions and parameters utilized in this experiment were as follows:
Column: Phenomenex Synergi 4μ Hydro-RP 80 Å, (ID 3.0 mm L 50 mm), Part Number 00B-4375-Y0, with Security Guard Cartridges AQ C18 4×3.0 mm, Part Number AJa-7511.
Column temperature: 30° C.
Autosampler temperature: Ambient (not lower than 20° C.)
Flow rate: 1 mL/min
UV detector: 260 nm
Injection volume: variable
Mobile phase A: 10 mM of ammonium phosphate (dibasic) in Milli-Q water and adjusted pH to 6.8 with Phosphoric acid.
Mobile phase B: 100% Acetonitrile.

The gradient for the RPLC was as provided in Table 7 below:

TABLE 7

| | % Mobile | Mobile |
|---|---|---|
| | phase A | phase B |
| 0 min | 98% | 2% |
| 3 min | 80% | 20% |
| 3.5 min | 0% | 100% |
| 5.5 min | 0% | 100% |
| 5.6 min | 98% | 2% |
| 8 min | 98% | 2% |

The % purity of the nucleoside was determined by comparing peak retention times for the nucleoside to a USP standard and subtracting impurity peaks. The % recovery was determined by measuring the area under the peaks and comparing to a calibrated reference standard.

### Results

Table 8 shows the Gamma radiation stability in terms of % purity of the nucleoside active agent for the nucleoside active agent complexed with protamine and formulated in the SAM/BB/EtOH/PLGA (8/67/5/20, % w/w) vehicle. The retention time for the nucleoside active agent was about 2 min.

TABLE 8
Nucleoside Active Agent Complexed with Protamine
in SAIB/BB/EtOH/PLGA (8/67/5/20, % w/w) Vehicle
Following Gamma Irradiation at 25 kGy

| Time | % Purity* |
|---|---|
| T = 1 Week | 94-97 |
| T = 2 Weeks | 95-97 |
| T = 8 Weeks | 95-97 |
| T = 24 weeks | 94-96 |

*n = 3 and the values represent the minimum and maximum range

The results shown in Table 8 indicate that the nucleoside active agent complexed with protamine and suspended in the SAM/BB/EtOH/PLGA (8/67/5/20, % w/w) vehicle, retained stability out to 24 weeks following exposure to Gamma radiation at a dose of 25 kGy and storage at 2-8° C., as indicated by % purity.

Table 9 shows the Gamma radiation stability in terms of % recovery of the nucleoside active agent for a nucleoside active agent complexed with protamine and suspended in a SAM/BB/EtOH/PLGA (8/67/5/20, % w/w) vehicle.

TABLE 9
Nucleoside Active Agent Complexed with Protamine
in SAIB/BB/EtOH/PLGA (8/67/5/20, % w/w) Vehicle
Following Gamma Irradiation at 25 kGy

| Time | % Recovery* |
|---|---|
| T = 1 Week | 99-103 |
| T = 2 Weeks | 101-104 |
| T = 8 Weeks | 90-93 |
| T = 24 weeks | 96-98 |

*n = 3 and the values represent the minimum and maximum % recovered

The results shown in Table 9 indicate that the nucleoside active agent complexed with protamine and suspended in the SAIB/BB/EtOH/PLGA (8/67/5/20, % w/w) vehicle, retained stability out to 24 weeks following exposure to Gamma radiation at a dose of 25 kGy and storage at 2-8° C., as indicated by % recovery.

### Example 3: Stability of Radiation-Sterilized Peptide Formulations

### Materials and Methods

A glucagon-like peptide-1 (GLP-1) analogue was prepared as (1) a solid powder, (2) a stock solution in water and (3) a lyophilized powder including GLP-1 complexed with zinc and protamine. The ratio of GLP-1 analogue to zinc in the complexed powder was 1:1, and the ratio of GLP-1 analogue to protamine in the complexed powder was 3:1.

Prior to lyophilization to produce the complexed powder, a stock solution including the active agent, complexing agents, and various excipients was prepared as set forth in Table 10 below:

TABLE 10

| | | | | Amount |
|---|---|---|---|---|
| | | Volume | Amount | per mL |
| Stock Solution | Concentration | (mL) | (mg) | (mg) |
| GLP-1 analogue | | | 500 | 10.54 |
| Ammonium | 0.396 g in | 5 | 19.8 | 0.42 |
| Bicarbonate(pH ~7.89) | 100 mL water | | | |
| | (50 mM) | | | |
| Zinc Acetate•2H2O | 2.194 g in | 2.43 | 53.3 | 1.12 |
| | 100 mL water | | | |
| | (100 mM) | | | |
| Sucrose Solution | 10 mg in | 7.5 | 75 | 1.58 |
| | 1 mL water | | | |
| | (300 mM) | | | |
| Protamine Sulfate | 10 mg/mL | 30.5 | 305 | 6.43 |
| | in water | | | |
| Acetic Acid, glacial | | 2 | | |
| | Total | 47.4 | 953.1 | |
| % GLP-1 analogue | Expected | | 52.4% | |
| (wt/wt) | (theoretical) | | | |

The lyophilized powder was prepared using the lyophilization conditions set forth above in Table 6. Following lyophilization the complexed powder was suspended in two different vehicles as set forth below:

Vehicle 1: SAM/BB/BA/PLA (20/60/10/10, % w/w), wherein BA=benzyl alcohol; and

Vehicle 2: SAM/BB/NMP/PLA (20/60/10/10, % w/w), wherein NMP=N— methyl-2-pyrrolidone.

The PLA in each of Vehicles 1 and 2 was lactic acid initiated and had a weight average molecular weight of 15.2 kD.

These formulations were exposed to a 25 kGy Gamma radiation dose.

Following exposure to Gamma radiation, storage for 24 hours, and extraction as described above for Example 2, % recovery and % purity were determined via RPLC analysis (also as described above for Example 2).

### Results

Table 11 (below) shows the Gamma radiation stability in terms of % purity and % recovery of the GLP-1 analogue active agent following exposure to a 25 kGy Gamma radiation dose.

TABLE 11

| Drug | Form | % Recovery | % Purity |
|---|---|---|---|
| Peptide | Solid (powder) | <1 | ND |
| Peptide | Solution in water | <1 | ND |
| Peptide | Vehicle 1 | 93.2 | 99.4 |
| Peptide | Vehicle 2 | 98.4 | 99.7 |

The results shown in Table 11 indicate that a peptide active agent (GLP-1 analogue) complexed with protamine and zinc and suspended in either a SAIB/BB/BA/PLA (20/60/10/10, % w/w) vehicle or a SAIB/BB/NMP/PLA (20/60/10/10, % w/w) vehicle, is stable following exposure to Gamma radiation at a dose of 25 kGy and storage at 2-8° C., as indicated by % recovery and % purity. Uncomplexed peptide active agent in powder form and in solution was not stable following exposure to a 25 kGy dose of Gamma radiation.

### Example 4: Stability of Complexed and Uncomplexed Radiation-Sterilized hGH Formulations

Uncomplexed hGH formulations including sucrose and methionine in a BB/PLA (80/20) vehicle were compared with complexed hGH formulations including sucrose and methionine in a BB/PLA (80/20) vehicle, where the hGH was complexed with either Zn or Zn/protamine. The formulations were tested for stability following Gamma irradiation.

### Materials and Methods

100 mg of BresaGen hGH powder was placed in a 15 mL wide-mouth glass jar. 5.5 mL of 25 mM NH₄HCO₃ (pH˜7.5) solution was added and the mixture was stirred for 30 min at room temperature, 400 rpm until it became clear. 274.8 mg of sucrose in water solution was added. For formulations containing Zn, 0.5 mg of zinc acetate dehydrate solution in water was added. For formulations containing Zn/protamine, 0.5 mg of zinc acetate dehydrate solution in water was added along with 10.2 mg of protamine sulfate in water. 2 mg of polysorbate 20 in water solution was slowly added to form a white precipitate. The resulting suspension was stirred for 30 min to complete the complexation reaction. All of the powders were prepared with 38% w/w of methionine relative to the total protein weight which was added as a powder to protein powder or as a solution to a protein-complex suspension.

The spray-dry conditions were as follows: Inlet temperature set up: 140° C., Aspirator 100%, Pump: 13%, Nozzle Cleaner: 2 pulses per min. In a one-component system, spray-dried powders were placed in a vial targeting 50 mg of hGH per dose and 1 mL of BB/PLA (80/20, % w/w) was added and homogenized for 10 min as a final suspension. The vial containing the suspension was stoppered and sealed in an aluminum pouch. The final package was sent to Gamma-STAT facility of Sterigenics, Corona, Calif. in a refrigerated package. The final one-component package was exposed to ambient conditions (˜25° C.) and exposed to a Gamma radiation dose of 22.5-27.5 kGy (targeting 25 kGy). Samples of hGH powder formulation without complex were also exposed to the different doses of Gamma-radiation as controls.

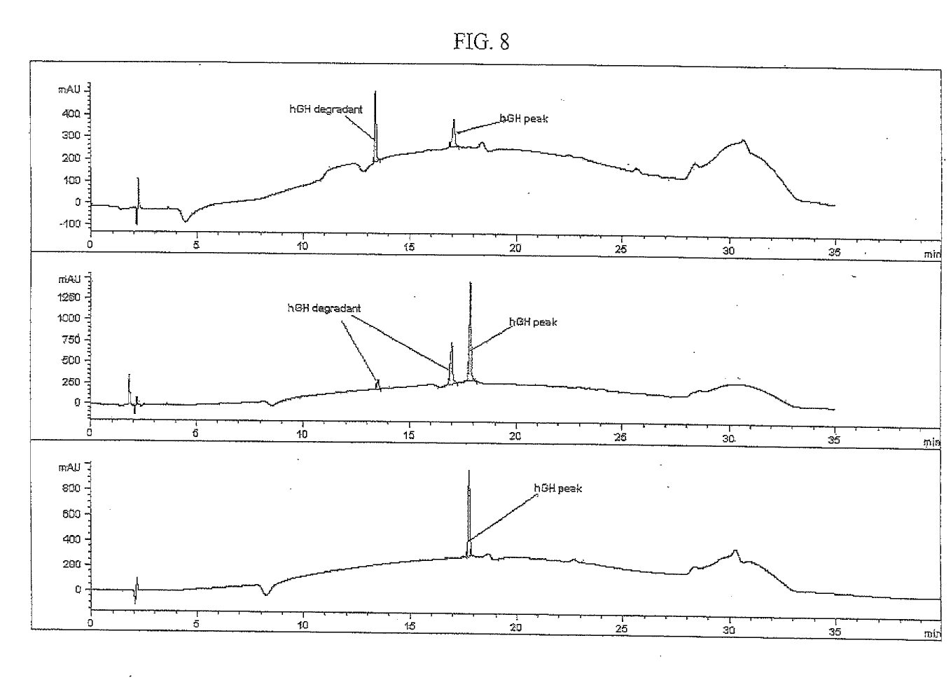

Stability of the above formulations was determined using RPLC analysis 24 hours after exposure to Gamma radiation. The results are shown in FIG. 8. Peak retention times were matched to a USP standard for hGH and impurity peaks were subtracted to obtain % purity levels.

Results

As shown in FIG. 8 and Table 12 (below), hGH complexed with Zn alone showed some improvement in stability following exposure to Gamma radiation over the uncomplexed control, i.e., 63% purity relative to 43% purity. Significant improvement in stability was seen for hGH complexed with Zn and protamine over the uncomplexed control, i.e., 92% purity relative to 43% purity.

TABLE 12

| | Vehicle, | |
|---|---|---|
| Drug | Form | % Purity |
| hGH + Sucrose + | BB/PLA | 43 |
| Methionine | (80/20), | |
| | suspension | |
| hGH + Zn + Sucrose + | BB/PLA | 63 |
| Methionine | (80/20), | |
| | suspension | |
| hGH + Zn/Protamine + | BB/PLA | 92 |
| Sucrose + Methionine | (80/20), | |
| | suspension | |

1. A composition, comprising:
a single-phase vehicle, comprising:
a biodegradable polymer in an amount ranging from about 5% to about 40% by weight of the vehicle, and
a hydrophobic solvent in an amount ranging from about 95% to about 60% by weight of the vehicle; and
an insoluble beneficial agent complex comprising a beneficial agent in the vehicle,
wherein the composition has been irradiated with ionizing radiation, and wherein the beneficial agent is present at a purity of about 90% or greater.

2. The composition of claim 1, wherein the ionizing radiation is selected from gamma radiation, e-beam radiation and x-ray radiation.

3. The composition of claim 1, wherein the beneficial agent maintains a purity of about 90% or greater when stored at 25° C. for a period of one month.

4. The composition of claim 1, wherein the ionizing radiation comprises a dose of about 10 kGy to about 25 kGy.

5. The composition of claim 1, wherein the insoluble beneficial agent complex comprises a peptide or a protein as the beneficial agent.

6. The composition of claim 1, wherein the ionizing radiation is gamma radiation.

7. The composition of claim 1, wherein the composition has a zero shear viscosity less than 1,200 centipoise at 25° C. and is not an emulsion, a gel or gel forming.

8. The composition of claim 1, wherein the insoluble beneficial agent complex comprises protamine.

9. The composition of claim 1, further comprising antioxidant.

10. The composition of claim 9, wherein the antioxidant is present in an amount ranging from about 1 wt % to about 45 wt %, relative to the amount of beneficial agent.

11. The composition of claim 1, further comprising methionine.

12. The composition of claim 1, wherein the insoluble beneficial agent complex comprises a beneficial agent having a molecular weight greater than 10 kD and less than 1000 kD.

13. The composition of claim 1, wherein the insoluble beneficial agent complex comprises a divalent metal salt of the beneficial agent.

14. The composition of claim 13, wherein the divalent metal is selected from Zn²⁺, Mg²⁺ and Ca²⁺.

15. The composition of claim 13, wherein the divalent metal is Zn²⁺.

16. The composition of claim 1, wherein the insoluble beneficial agent complex comprises beneficial agent and protamine in the form of particles, and wherein the particles further comprise bulking agent and surfactant.

17. (canceled)

18. (canceled)

19. The composition of claim 1, wherein the insoluble beneficial agent complex is dispersed in the vehicle in the form of particles having an average size ranging from about 1 μm to about 400 μm.

20. (canceled)

21. (canceled)

22. (canceled)

23. (canceled)

24. (canceled)

25. A method of making a composition, comprising:
combining a biodegradable polymer and a hydrophobic solvent to form a single-phase vehicle of the composition,
wherein the biodegradable polymer is included in an amount of from about 5% to about 40% by weight of the vehicle, and
the hydrophobic solvent is included in an amount of from about 95% to about 60% by weight of the vehicle; and
dispersing an insoluble beneficial agent complex comprising a beneficial agent in the vehicle to form the composition, and
irradiating the composition with ionizing radiation, wherein the beneficial agent maintains a purity of about 90% or greater when stored at 25° C. for a period of 24 hours after irradiation.

26. (canceled)

27. (canceled)

28. A method of administering a beneficial agent to a subject, comprising:
administering to the subject via injection a sterile, irradiated composition comprising
a vehicle comprising
a biodegradable polymer present in an amount of from about 5% to about 40% by weight of the vehicle, and
a hydrophobic solvent present in an amount of from about 95% to about 60% by weight of the vehicle; and
an insoluble beneficial agent complex dispersed in the vehicle,
wherein the composition has a zero shear viscosity less than 1,200 centipoise at 25° C. and is not an emulsion,
wherein the beneficial agent has a purity of at least 90% or greater.

29. (canceled)
